(19) United States
(12) Patent Application Publication
(10) Pub. No.: US 2026/0159503 A1
(43) Pub. Date: Jun. 11, 2026

(54) 1,2,3-TRIAZOLE LINKER-CONTAINING COMPOUNDS

(71) Applicant: HIGH POINT UNIVERSITY, High Point, NC (US)

(72) Inventor: Comfort Ahenkan Boateng, High Point, NC (US)

(21) Appl. No.: 19/411,133

(22) Filed: Dec. 5, 2025

Related U.S. Application Data
(60) Provisional application No. 63/728,367, filed on Dec. 5, 2024.

(51) Int. Cl. C07D 401/14; A61K 31/454; A61K 31/496; A61K 31/506; A61P 25/00; C07D 401/06; C07D 403/06; C07D 403/14

(52) U.S. Cl. CPC C07D 401/14; A61K 31/454; A61K 31/496; A61K 31/506; A61P 25/00; C07D 401/06; C07D 403/06; C07D 403/14

(57) ABSTRACT

The disclosure provides compounds which exhibit dopamine receptor partial agonist or antagonist activity and which may be useful as therapeutic agents for the treatment of diseases and disorders associated with dopamine receptors. Also provided are methods for the preparation of such compounds, pharmaceutical compositions comprising the disclosed compounds, and methods for treating diseases or disorders with such compounds or pharmaceutical compositions.

### FEDERALLY SPONSORED RESEARCH OR DEVELOPMENT

This invention was made with government support under Grants DPIDA058385 and R21DA050896, awarded by the National Institutes of Health. The government has certain rights in the invention.

### CROSS-REFERENCE TO RELATED APPLICATIONS

The present application claims priority to U.S. Provisional Patent Application No. 63,728,367, filed Dec. 5, 2024, the disclosure of which is incorporated herein by reference in its entirety.

### FIELD OF THE INVENTION

The present application is directed to compounds, methods, and compositions for either inhibiting or activating certain dopamine receptors. Such compounds, methods, and compositions may find use, in particular, for treating neuropsychiatric disorders.

### BACKGROUND OF THE INVENTION

The catecholamine neurotransmitter dopamine (DA) signals by binding and activating dopamine receptors (DRs), a family of G protein-coupled receptors (GPCRs). DRs are subcategorized on the basis of signaling and sequence homology into the excitatory D₁-like receptors, which includes dopamine D₁ and D₅ receptors (D₁R, D₅R), and the inhibitory D₂-like receptors, which includes doplamine D₂, D₃, and D₄ receptors (D₂R, D₃R, and D₄R). All D₂-like receptors share a similar signaling mechanism, coupling to the Gα_{i/o} G proteins and recruiting β-arrestin. They also share substantial amino acid sequence homology in their orthosteric binding sites. However, they vary in their expression patterns within the brain and in their synaptic localization.

D₄Rs are mainly found in the hippocampal (HC) and prefrontal cortical (PFC) regions of the brain with a lower overall level of expression compared to D₂Rs and D₃Rs, which are located primarily in the basal ganglia, striatum, and pituitary gland. Drugs targeting D₂Rs and D₃Rs can alter locomotor function and motivated states, and D₂Rs are a primary target for antipsychotic drugs. In contrast, the activity of D₄Rs located in the HC and the PFC influence exploratory behavior, attention, and performance in cognitive tasks such as novel object recognition and inhibitory avoidance. Activating D₄Rs could be a route for a potential treatment for cognitive deficits associated with attention-deficit/hyperactivity disorder (ADHD) and schizophrenia. Preclinical studies showed D₄R agonists improved performance cognitive tasks, such as novel object recognition tasks, 5-trial repeated acquisition inhibitory avoidance tasks, and social recognition tasks. Recent studies indicate that pharmacological activation of D₄Rs could also minimize the negative effects of opioid drugs such as morphine. Antagonizing D₄Rs might be helpful in treating L-DOPA-induced dyskinesias and substance use disorders (SUDs), particularly psychostimulant use disorders.

Despite the clinical significance, there are currently no FDA-approved medications for treating psychostimulant use disorders, nor are there FDA-approved medications that selectively target D₄R. Such compounds would be advantageous for treatment of a range of diseases and disorders.

### SUMMARY OF THE INVENTION

This present disclosure relates to compounds, compositions, and methods involving such compounds and compositions to treat disorders and diseases associated with the central nervous system (CNS) and, particularly, to such disorders and diseases in humans. In particular, the compounds, compositions, and methods provided herein relate to the antagonism of dopamine receptors and, as such, use of the disclosed compounds may be advantageous for treatment of a diverse array of complex pathologies, including, e.g., cognitive disorders and substance use disorders (e.g., relating to cocaine addiction).

Various compounds of the disclosure comprise a 1, 2, 3-triazole linker moiety; certain such compounds are analogues of known compounds wherein an amide linker of the known compounds is replaced with the 1,2,3-triazole linker moiety. In some embodiments, the 1,2,3-triazole linker-containing compounds exhibit high affinity and/or selectivity for the dopamine D₄R receptor (D₄R). In a further aspect of the disclosure is provided a pharmaceutical composition comprising a compound as described herein and one or more pharmaceutically acceptable carriers. The compound and one or more pharmaceutically acceptable carriers can be, e.g., in the form of a mixture of components and can be formulated into various dosage forms (e.g., tablets, caplets, gels, solutions, suspensions, and the like).

The disclosure further provides a method for treating a disease or disorder associated with the central nervous system (CNS), comprising administering a therapeutically effective amount of a compound as provided herein or a pharmaceutical composition as provided herein.

This disclosure includes, without limitation, the following embodiments:
Embodiment 1: A compound of Formula 1:

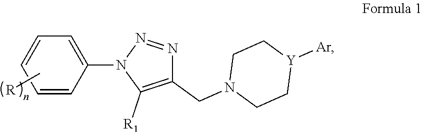

wherein:
R is a halogen, hydroxyl, C1-6 alkyl, or C1-6 alkoxy, wherein the C1-6 alkyl or C1-6 alkoxy can be optionally substituted with one or more substituents selected from the group consisting of halogen, optionally substituted C1-6 alkyl, optionally substituted C1-6 alkenyl, OR₂, and N(R₂)₂;
n is an integer from 0 to 5;
R₁ is H, C1-6 alkyl, aryl, or heteroaryl, wherein the alkyl, aryl, or heteroaryl is optionally substituted with one or more substituents selected from the group consisting of halogen, optionally substituted C1-6 alkyl, optionally substituted C1-6 alkenyl, OR₃, and N(R₂)₂;
Y is N or CH;
Ar is aryl or heteroaryl, wherein the aryl or heteroaryl is optionally substituted with one or more substituents selected from the group consisting of halogen, optionally substituted C1-6 alkyl, optionally substituted C1-6 alkenyl, OR₂, and N(R₂)₂;
R₂ is selected from H and C1-6 alkyl; and
R₃ is selected from H, optionally substituted C1-6 alkyl, and optionally substituted C1-6 aralkyl.
Embodiment 2: The compound of Embodiment 1, wherein n=0.
Embodiment 3: The compound of Embodiment 1, wherein n=1.
Embodiment 4: The compound of Embodiment 1 or 3, wherein R is an optionally substituted C1-6 alkyl or optionally substituted C1-3 alkyl or optionally substituted C1-2 alkyl (e.g., CH₃, CF₃, or CH₂CH₃).
Embodiment 5: The compound of Embodiment 1 or 3, wherein R is C1-6 alkyl or C1-3 alkyl or C1-2 alkyl (e.g., CH₃ or CH₂CH₃).
Embodiment 6: The compound of Embodiment 1 or 3, wherein R is a halogen (e.g., Cl or F), hydroxyl (—OH), or optionally substituted C1-6 alkoxy or optionally substituted C1-3 alkoxy (e.g., OCH₃).
Embodiment 7: The compound of any of Embodiments 1-6, wherein Y is CH.
Embodiment 8: The compound of any of Embodiments 1-6, wherein Y is N.
Embodiment 9: The compound of any of Embodiments 1-8, wherein Ar is aryl.
Embodiment 10: The compound of any of Embodiments 1-8, wherein Ar is heteroaryl.
Embodiment 11: The compound of any of Embodiments 1-8, wherein Ar is an optionally substituted phenyl ring.
Embodiment 12: The compound of any of Embodiments 1-8, wherein Ar is an optionally substituted pyridine ring (e.g., an optionally substituted 2-pyridinyl).
Embodiment 13: The compound of any of Embodiments 1-8, wherein Ar is an optionally substituted pyrimidine ring (e.g., an optionally substituted 2-pyrimidinyl).
Embodiment 14: The compound of any of Embodiments 1-8, wherein Ar is an optionally substituted naphthyl group.
Embodiment 15: The compound of any of Embodiments 1-14, wherein Ar comprises no substituent.
Embodiment 16: The compound of any of Embodiments 1-14, wherein Ar comprises one substituent.
Embodiment 17: The compound of any of Embodiments 1-14 or 16, wherein Ar comprises a Cl or F substituent.
Embodiment 18: The compound of any of Embodiments 1-14 or 16 or 17, wherein Ar comprises a methyl substituent.
Embodiment 19: The compound of any of Embodiments 1-18, wherein R₁ is H.
Embodiment 20: The compound of any of Embodiments 1-18, wherein R₁ is

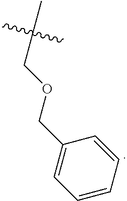 [SMILES: *COCc1ccccc1]

Embodiment 21: The compound of any of Embodiments 1, 3, 4, 5, 8, 10, 12, 16, 17, and 19 wherein R is CH₃, Y is N, R₁ is H, and Ar is a substituted pyridine ring.
Embodiment 22: The compound of Embodiment 1, selected from the group consisting of:

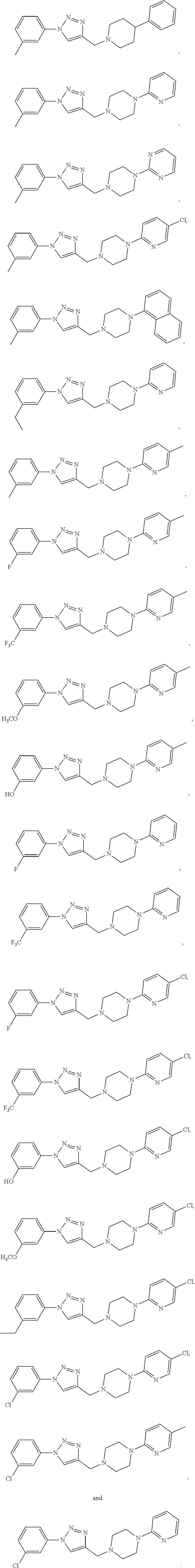

Embodiment 23: The compound of Embodiment 1, selected from the group consisting of:

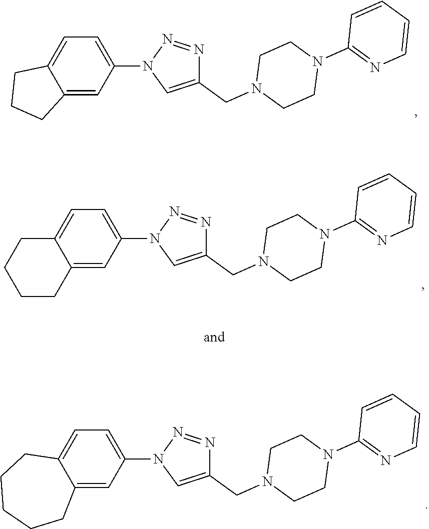 [SMILES: c1ccc(N2CCN(Cc3cn(-c4ccc5c(c4)CCC5)nn3)CC2)nc1 ; c1ccc(N2CCN(Cc3cn(-c4ccc5c(c4)CCCC5)nn3)CC2)nc1 ; c1ccc(N2CCN(Cc3cn(-c4ccc5c(c4)CCCCC5)nn3)CC2)nc1]

Embodiment 24: A pharmaceutical composition comprising the compound of any of Embodiments 1-23 and one or more pharmaceutically acceptable carriers.
Embodiment 25: A method for treating a disease or disorder associated with the central nervous system, comprising administering a therapeutically effective amount of the compound of any of Embodiments 1-23 or the pharmaceutical composition of Embodiment 24.
Embodiment 26: The method of Embodiment 25, wherein the disease or disorder is a cognitive deficit associated with neuropsychiatric disorders, e.g., schizophrenia and/or attention deficit/hyperactivity disorder (ADHD).
Embodiment 27: The method of Embodiment 25, wherein the disease or disorder is L-DOPA-induced dyskinesias or an impulse-control disorder, e.g., substance abuse disorder, eating disorder, or pathological gambling.
Embodiment 28: The method of Embodiment 27, wherein the substance abuse disorder is cocaine addiction.
Embodiment 29: The method of Embodiment 27, wherein the substance abuse disorder is opioid addiction.

These and other features, aspects and advantages of the disclosure will be apparent from a reading of the following detailed description together with the accompanying drawings, which are briefly described below. The invention includes any combination of two, three, four, or more of the above-noted embodiments as well as a combination of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

In order to provide an understanding of embodiments of the invention, reference is made to the appendix drawings, which are not necessarily drawn to scale, and in which reference to components of exemplary only and should not be construed as limiting the invention.

FIG. 1 is a table of D₂R-, D₃R-, and D₄R-mediated β-arrestin recruitment data for various compounds according to certain, non-limiting embodiments of the present disclosure;

FIG. 2. is an image of the D₄R surface and aerial view of the binding pocket docked with non-limiting matching representatives of triazole-based (17; top inset) and amide-based (2; bottom inset) analog sets according to certain non-limiting embodiments of the present disclosure;

FIG. 3(A) is an image of a non-limiting example compound (Compound 17) in complex with D₄R and FIG. 3(B) is an image of a non-limiting comparative compound (Compound 2) in complex with D₄R;

FIG. 4(A) is an image of interactions in the D₄R for a non-limiting example compound (Compound 17) and FIG. 4(B) is a corresponding image for a non-limiting comparative compound (Compound 2);

FIG. 5(A) is an image of interactions in the D₄R extended-binding pocket (EBP) for a non-limiting example compound (Compound 17) and FIG. 5(B) is a corresponding image for a non-limiting comparative compound (Compound 2);

FIG. 6(A) is an image of a binding pose for a non-limiting example compound (Compound 14) in the “opposite pose” and FIG. 6(B) is an image of a binding pose for Compound 14 in the “consistent pose”;

FIG. 7(A) is an image of a binding pose for a non-limiting comparative compound (Compound 7) in the “opposite pose” and FIG. 7(B) is an image of a binding pose for Compound 7 in the “consistent pose”;

FIG. 8 provides phase I metabolic stability of non-limiting comparative compounds 2-7 and non-limiting example compounds 14-19 in rat liver microsomes, where 8A, 8B, 8C, 8D, 8E, and 8F provide data presented as percent compound remaining (means±SEM) at 0-, 30-, and 60-min following incubation with rat liver microsomes in the presence of NADPH; 8G provides pairwise comparison of calculated compound half-lives for each amide-triazole analog pair; and 8H provides calculated half-lives for each compound, expressed as means±SD, n=3;

FIG. 9 provides phase I metabolic stability of non-limiting comparative compounds 2-7 and non-limiting example compounds 14-19 in human liver microsomes, where 9A, 9B, 9C, 9D, 9E, and 9F provide data presented as percent compound remaining (means±SEM) at 0-, 30-, and 60-min following incubation with human liver microsomes in the presence of NADPH; 9G provides pairwise comparison of calculated compound half-lives for each amide-triazole analog pair; and 9H provides calculated half-lives for each compound, expressed as means±SD, n=3;

FIGS. 10A and 10B provide non-phase I metabolic stability of non-limiting comparative compounds 2-7 and non-limiting example compounds 14-19 in rat liver microsomes and FIGS. 10C and 10D provide non-phase I metabolic stability of non-limiting comparative compounds 2-7 and non-limiting example compounds 14-18 in human liver microsomes; data are presented as percent compound remaining (means±SEM) at 0-, 30-, and 60-min following incubation with rat or human liver microsomes in the absence of NADPH;

FIGS. 11A, 11B, 11C, and 11D are plots of time-dependent in vivo pharmacokinetic analyses of non-limiting example compounds 14, 17, 15, and 18, respectively, in Sprague Dawley (SD) rats following intraperitoneal (i.p.) administration of 10 mg/kg of each compound, with Data expressed as means±SEM, n=3 for each time point;

FIG. 12 is a table of data for certain non-limiting compounds of the present disclosure;

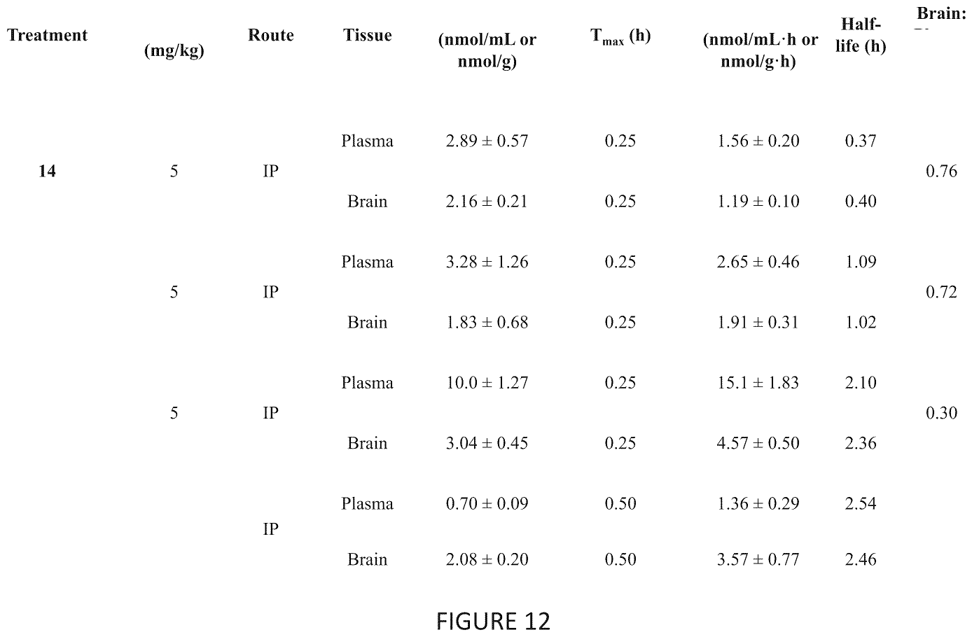

FIGS. 13A, 13B, 13C, and 13D show effects of Compound 15 (FMJ-01-38) and Compound 17 (FMJ-01-54) on locomotor activity in adolescent SHR/NCrl and Wistar rats; FIG. 13A and FIG. 13C show total distance moved (cm), and FIG. 13B and FIG. 13D show movement duration(s) in the open-field test, with data expressed as mean±SEM (n=12 per group). ###p<0.001; ***p<0.001, **p<0.01 vs. SHR/NCrl control (vehicle-treated) rats;

FIGS. 14A, 14B, 14C, and 14D show effects of Compound 15 (FMJ-01-38) and Compound 17 (FMJ-01-54) on inattention-like behavior in adolescent SHR/NCrl rats; FIG. 14A and FIG. 14C show percent spontaneous alternation, and FIG. 14B and FIG. 14D show total arm entries in the 8-minute Y-maze test, with data expressed as mean±SEM (n=12 per group). ##p<0.01; ***p<0.001; *p<0.05 vs. SHR/NCrl control (vehicle-treated) rats; $p<0.05 vs. Wistar control (vehicle-treated) rats;

FIGS. 15A and 15B show effects of Compound 15 (FMJ-01-38) and Compound 17 (FMJ-01-54) on attention/recognition memory in adolescent SHR/NCrl and Wistar rats; percent preference for the novel object (FIGS. 15A and 15B) is shown, with data are expressed as mean±SEM (n=12 per group). ##p<0.01; ***p<0.001; **p<0.01, *p<0.05 vs. SHR/NCrl control (vehicle-treated) rats; $$p<0.01 vs Wistar control (vehicle-treated) rats;

FIGS. 16A, 16B, 16C, and 16D show effects of Compound 15 (FMJ-01-38) and Compound 17 (FMJ-01-54) on impulsive-like behavior in adolescent SHR/NCrl rats; FIGS. 16A and 16C show the percentage choice for the larger delayed reward, and FIGS. 16B and 16D show the number of nonreinforced responses for the immediate small reward in the delay-discounting task, with data expressed as mean±SEM (n=10 per group); *p<0.05 vs. SHR/NCrl control (vehicle-treated) rats; and

FIGS. 17A, 17B, 17C, and 17D show the effects of Compound 15 (FMJ-01-38) and Compound 17 (FMJ-01-54) on impulsive-like behavior in adolescent Wistar rats; FIGS. 17A and 17C show the percentage choice for the larger delayed reward, and FIGS. 17B and 17D show the number of nonreinforced responses for the immediate small reward in the delay-discounting task, with data expressed as mean±SEM (n=10 per group); *p<0.05 vs. Wistar control (vehicle-treated) rats.

### DETAILED DESCRIPTION

The present invention now will be described more fully hereinafter. However, the invention may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

The present disclosure provides compounds that may function as partial agonists or antagonists at dopamine receptors, as well as pharmaceutical compositions thereof. The disclosure also provides methods for using such compounds to treat a variety of diseases or disorders that may be responsive to the antagonism of dopamine receptors. Several lines of evidence indicate that pharmacological targeting of dopamine receptors (and, in particular, the D₄R subtype) may be advantageous for a diverse array of complex pathologies, including substance use disorders and cognitive disorders. In some embodiments, the compositions and methods can be used in the treatment of addiction. Treatment can comprise the use of a compound of the present disclosure as a single active agent. In other embodiments, treatment can comprise the use of a compound of the present disclosure in combination with one or more further active agents. Specific pharmaceutical compositions and methods of treatment are further described below.

### Compounds

In one aspect of the disclosure is provided a compound of Formula 1:

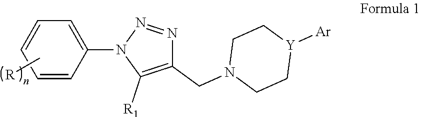

wherein:
R is a halogen, OR₂ (e.g., hydroxyl or C1-6 alkoxy), or C1-6 alkyl, wherein the R₂ or C1-6 alkyl is optionally substituted with one or more substituents selected from the group consisting of halogen, optionally substituted C1-6 alkyl, optionally substituted C1-6 alkenyl, OR₂, and N(R₂)₂;
n is an integer from 0 to 5;
R₁ is H, C1-6 alkyl, aryl, or heteroaryl, wherein the alkyl, aryl, or heteroaryl is optionally substituted with one or more substituents selected from the group consisting of halogen, optionally substituted C1-6 alkyl, optionally substituted C1-6 alkenyl, OR₃, and N(R₂)₂;
Y is N or CH;
Ar is aryl or heteroaryl, wherein the aryl or heteroaryl is optionally substituted with one or more substituents selected from the group consisting of halogen, optionally substituted C1-6 alkyl, optionally substituted C1-6 alkenyl, OR₂, and N(R₂)₂;
R₂ is selected from H and C1-6 alkyl; and
R₃ is selected from H, optionally substituted C1-6 alkyl, and optionally substituted C1-6 aralkyl.

The term “alkyl” as used herein means saturated straight, branched, or cyclic hydrocarbon groups (i.e., cycloalkyl groups). In particular embodiments, alkyl refers to groups comprising 1 to 10 carbon atoms (“C1-10 alkyl”). In further embodiments, alkyl refers to groups comprising 1 to 8 carbon atoms (“C1-8 alkyl”), 1 to 6 carbon atoms (“C1-6 alkyl”), or 1 to 4 carbon atoms (“C1-4 alkyl”). In other embodiments, alkyl refers to groups comprising 3-10 carbon atoms (“C3-10 alkyl”), 3-8 carbon atoms (“C3-8 alkyl”), or 3-6 carbon atoms (“C3-6 alkyl”). In specific embodiments, alkyl refers to methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl.

The term “heteroalkyl” as used herein means an alkyl group, having at least one atom within the chain which is not carbon, and includes heterocycloalkyl groups. Preferred heteroatoms include sulfur, oxygen, and nitrogen. “Aralkyl” refers to a radical of the formula —R^{c}-aryl where R^{c} is an alkylene chain as defined above, for example, methylene, ethylene, and the like.

“Optionally substituted” in reference to a substituent group refers to substituent groups optionally substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃); hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “alkoxy” as used herein means straight or branched chain alkyl groups linked by an oxygen atom (i.e., —O-alkyl), wherein alkyl is as described above. In particular embodiments, alkoxy refers to oxygen-linked groups comprising 1 to 10 carbon atoms (“C1-10 alkoxy”). In further embodiments, alkoxy refers to oxygen-linked groups comprising 1 to 8 carbon atoms (“C1-8 alkoxy”), 1 to 6 carbon atoms (“C1-6 alkoxy”), 1 to 4 carbon atoms (“C1-4 alkoxy”) or 1 to 3 carbon atoms (“C1-3 alkoxy”).

The term “heterocycloalkyl” means a non-aromatic, monocyclic or polycyclic ring comprising, in addition to carbon and hydrogen atoms, at least one atom within the chain which is not carbon. Preferred heteroatoms include sulfur, oxygen, and nitrogen.

The term “halo” or “halogen” as used herein means fluorine, chlorine, bromine, or iodine.

The term “alkylthio” as used herein means a thio group with one or more alkyl substituents, where alkyl is defined as above.

The term “amino” as used herein means a moiety represented by the structure NR₂, and includes primary amines, and secondary and tertiary amines substituted by alkyl or aryl (i.e., alkylamino or arylamino, respectively). Thus, R₂ may represent two hydrogen atoms, two alkyl moieties, two aryl moieties, one aryl moiety and one alkyl moiety, one hydrogen atom and one alkyl moiety, or one hydrogen atom and one aryl moiety.

The term “cycloalkyl” means a non-aromatic, monocyclic or polycyclic ring comprising carbon and hydrogen atoms.

The term “aryl” as used herein means a stable monocyclic, bicyclic, or tricyclic carbon ring of up to 8 members in each ring, wherein at least one ring is aromatic as defined by the Hückel 4n+2 rule. Exemplary aryl groups include, but are not limited to, phenyl, naphthyl, tetrahydronaphthyl, and biphenyl.

The term “heteroaryl” as used herein refers to an aryl group wherein at least one of the carbon atoms of the ring is substituted with a heteroatom, wherein each heteroatom may be selected from N, O, and S. As used herein, the heteroaryl ring may be selected from monocyclic or bicyclic and fused or bridged ring systems, wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, pyridinyl, and pyrimidinyl.

When stereochemistry is not specified, certain molecules described herein include isomers, such as enantiomers and diastereomers, mixtures of enantiomers, including racemates, mixtures of diastereomers, and other mixtures thereof, to the extent they can be made by one of ordinary skill in the art by routine experimentation. In certain embodiments, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration.

In certain embodiments, chiral centers in compounds of the present disclosure may have the S or R configuration as defined by the IUPAC 1974 Recommendations.

In some embodiments of Formula 1, Ar is a monocyclic or bicyclic aryl or heteroaryl group (e.g., a fused bicyclic group). In some embodiments of Formula 1, Ar is an optionally substituted aryl or an optionally substituted nitrogen-containing heteroaryl. In some embodiments, Ar is an optionally substituted pyridinyl or pyrimidinyl substituent, e.g., an optionally substituted 2-pyridinyl or 2-pyrimidinyl substituent. In some embodiments, Ar comprises an optionally substituted phenyl ring fused to an optionally substituted cycloalkyl group, i.e., a benzocycloalkane.

In some embodiments of Formula 1, R is an electron withdrawing group; in some embodiments, R is an electron donating group. In some embodiments of Formula 1, at least one optional substituent on Ar is present. In some such embodiments, the substituent is an electron withdrawing group and in some embodiments, the substituent is an electron donating group.

In some embodiments of Formula 1, R₁ is H. In some embodiments, R₁ is alkyl. In some embodiments, R₁ is alkyl (e.g., CH₃) substituted with OR₃, e.g., where R₃ is alkyl (e.g., CH₃ or CH₂CH₃) or aralkyl (e.g., CH₂-phenyl). In one particular embodiment, R₁ is:

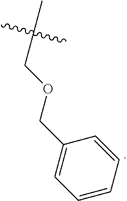 [SMILES: *COCc1ccccc1]

In one aspect of the disclosure is provided a compound of Formula 1a:

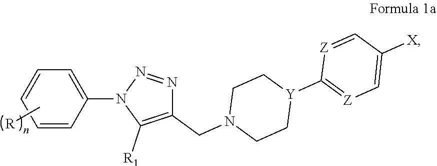

wherein:
   substituents R, n, R₁, and Y are as defined for Formula 1 above,
   Z and Z′ are each independently selected from N and CH; and
   X is selected from the group consisting of halogen, optionally substituted C1-6 alkyl, optionally substituted C1-6 alkenyl, OR₂, and N(R₂)₂.

In another aspect of the disclosure is provided a compound of Formula 1b:

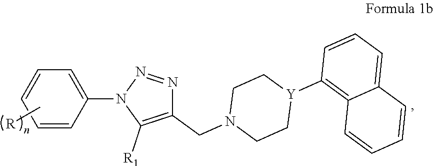

wherein:
   substituents R, n, R₁, and Y are as defined for Formula 1 above.

Certain example compounds provided herein include, but are not limited to,
4-phenyl-1-((1-(m-tolyl)-1H-1,2,3-triazol-4-yl)methyl)piperidine;
1-(pyridin-2-yl)-4-((1-(m-tolyl)-1H-1,2,3-triazol-4-yl)methyl)piperazine;
2-(4-((1-(m-tolyl)-1H-1,2,3-triazol-4-yl)methyl)piperazin-1-yl)pyrimidine;
1-(5-chloropyridin-2-yl)-4-((1-(m-tolyl)-1H-1,2,3-triazol-4-yl)methyl)piperazine; and
1-((1-(3-ethylphenyl)-1H-1,2,3-triazol-4-yl)methyl)-4-(pyridin-2-yl)piperazine.

Certain compounds are as represented in Table 1 provided herein below, along with Comparative examples as mentioned in the Examples section.

TABLE 1
Non-limiting Compounds of Formula 1

| Compound No. | Structure |
|---|---|
| 1 (Comparative) | 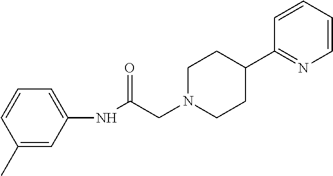 [SMILES: Cc1cccc(NC(=O)CN2CCC(c3ccccn3)CC2)c1] |
| 5 (Comparative) | 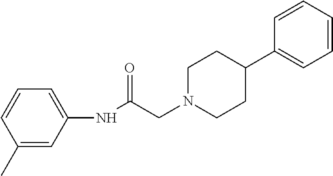 [SMILES: Cc1cccc(NC(=O)CN2CCC(c3ccccc3)CC2)c1] |
| 14 | 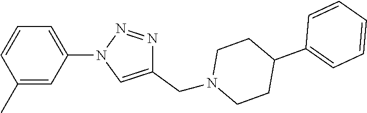 [SMILES: Cc1cccc(-n2cc(CN3CCC(c4ccccc4)CC3)nn2)c1] |
| 2 (Comparative) | 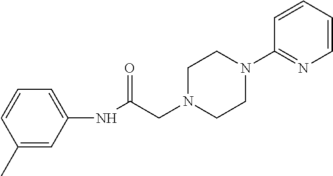 [SMILES: Cc1cccc(NC(=O)CN2CCN(c3ccccn3)CC2)c1] |
| 15 | 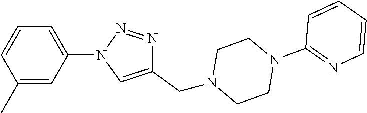 [SMILES: Cc1cccc(-n2cc(CN3CCN(c4ccccn4)CC3)nn2)c1] |
| 4 (Comparative) | 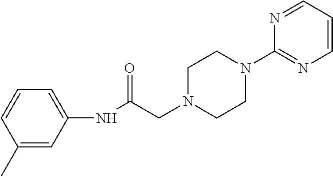 [SMILES: Cc1cccc(NC(=O)CN2CCN(c3ncccn3)CC2)c1] |
| 16 | 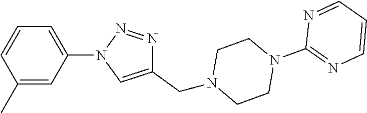 [SMILES: Cc1cccc(-n2cc(CN3CCN(c4ncccn4)CC3)nn2)c1] |
| 7 (Comparative) | 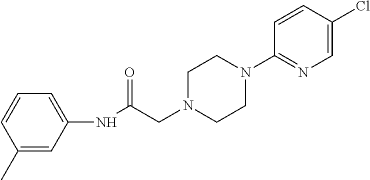 [SMILES: Cc1cccc(NC(=O)CN2CCN(c3ccc(Cl)cn3)CC2)c1] |
| 17 | 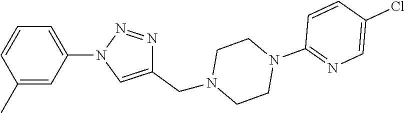 [SMILES: Cc1cccc(-n2cc(CN3CCN(c4ccc(Cl)cn4)CC3)nn2)c1] |
| 6 (Comparative) | 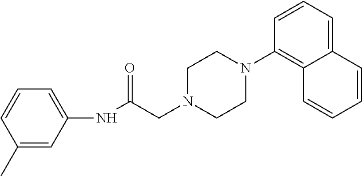 [SMILES: Cc1cccc(NC(=O)CN2CCN(c3cccc4ccccc34)CC2)c1] |
| 18 | 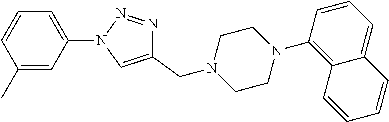 [SMILES: Cc1cccc(-n2cc(CN3CCN(c4cccc5ccccc45)CC3)nn2)c1] |
| 3 (Comparative) | 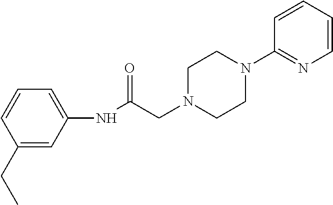 [SMILES: CCc1cccc(NC(=O)CN2CCN(c3ccccn3)CC2)c1] |
| 19 | 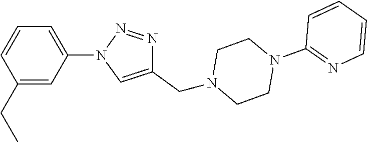 [SMILES: CCc1cccc(-n2cc(CN3CCN(c4ccccn4)CC3)nn2)c1] |
| 20 | 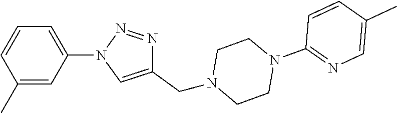 [SMILES: Cc1ccc(N2CCN(Cc3cn(-c4cccc(C)c4)nn3)CC2)nc1] |
| 21 | 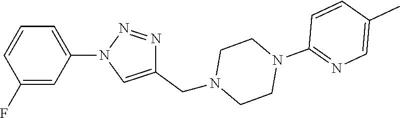 [SMILES: Cc1ccc(N2CCN(Cc3cn(-c4cccc(F)c4)nn3)CC2)nc1] |
| 22 | 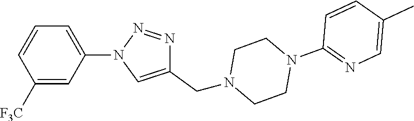 [SMILES: Cc1ccc(N2CCN(Cc3cn(-c4cccc(C(F)(F)F)c4)nn3)CC2)nc1] |
| 23 | 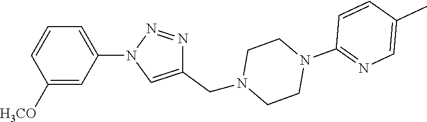 [SMILES: COc1cccc(-n2cc(CN3CCN(c4ccc(C)cn4)CC3)nn2)c1] |
| 24 | 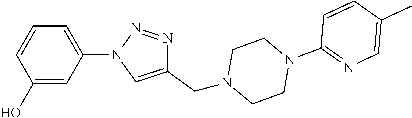 [SMILES: Cc1ccc(N2CCN(Cc3cn(-c4cccc(O)c4)nn3)CC2)nc1] |
| 25 | 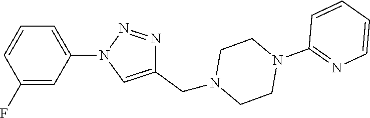 [SMILES: Fc1cccc(-n2cc(CN3CCN(c4ccccn4)CC3)nn2)c1] |
| 26 | 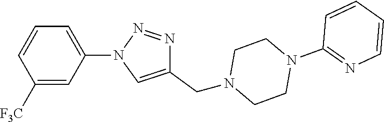 [SMILES: FC(F)(F)c1cccc(-n2cc(CN3CCN(c4ccccn4)CC3)nn2)c1] |
| 27 | 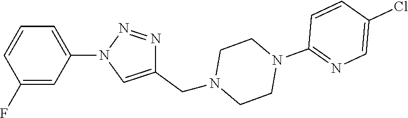 [SMILES: Fc1cccc(-n2cc(CN3CCN(c4ccc(Cl)cn4)CC3)nn2)c1] |
| 28 | 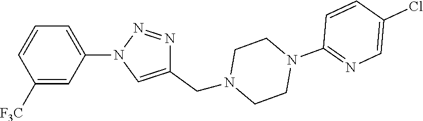 [SMILES: FC(F)(F)c1cccc(-n2cc(CN3CCN(c4ccc(Cl)cn4)CC3)nn2)c1] |
| 29 | 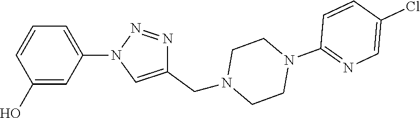 [SMILES: Oc1cccc(-n2cc(CN3CCN(c4ccc(Cl)cn4)CC3)nn2)c1] |
| 30 | 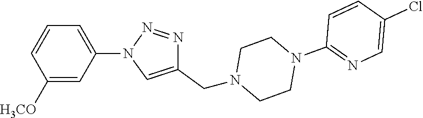 [SMILES: COc1cccc(-n2cc(CN3CCN(c4ccc(Cl)cn4)CC3)nn2)c1] |
| 31 | 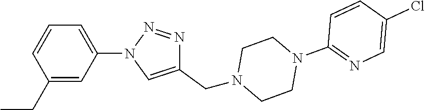 [SMILES: CCc1cccc(-n2cc(CN3CCN(c4ccc(Cl)cn4)CC3)nn2)c1] |
| 32 | 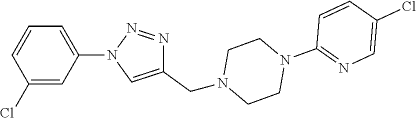 [SMILES: Clc1ccc(N2CCN(Cc3cn(-c4cccc(Cl)c4)nn3)CC2)nc1] |
| 33 | 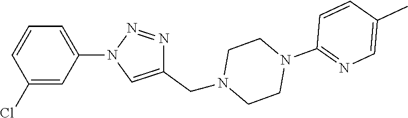 [SMILES: Cc1ccc(N2CCN(Cc3cn(-c4cccc(Cl)c4)nn3)CC2)nc1] |
| 35 | 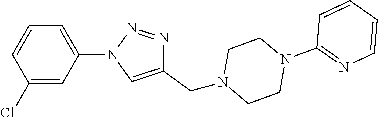 [SMILES: Clc1cccc(-n2cc(CN3CCN(c4ccccn4)CC3)nn2)c1] |
| 36 | 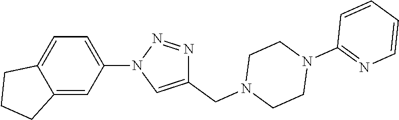 [SMILES: c1ccc(N2CCN(Cc3cn(-c4ccc5c(c4)CCC5)nn3)CC2)nc1] |
| 37 | 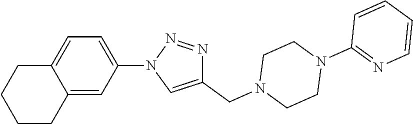 [SMILES: c1ccc(N2CCN(Cc3cn(-c4ccc5c(c4)CCCC5)nn3)CC2)nc1] |
| 38 | 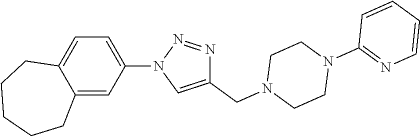 [SMILES: c1ccc(N2CCN(Cc3cn(-c4ccc5c(c4)CCCCC5)nn3)CC2)nc1] |

The disclosed compounds can exhibit activity at the dopamine (DA) receptors and, in some embodiments, exhibit affinity and/or selectivity specifically for the D₄R receptor (e.g., over D₂R and D₃R). In some embodiments, the disclosed compounds address issues found in follow-up behavioral studies with lead compounds from a corresponding series comprising an amide linker in place of the disclosed 1,2,3-triazole-linker (in Keck, T. M. et al., J. Med. Chem. 2019, 62, 3722-3740, which is incorporated herein by reference in its entirety). The amide linker-containing compounds exhibited pharmacokinetic limitations, confirmed using in vitro pharmacokinetic studies that determined that the structural template was labile, with the amide linker consistently identified as the key site of both Phase I and non-Phase I metabolism.

Advantageously, in some embodiments, the disclosed compounds exhibit high D₄R affinity and selectivity while improving the pharmacokinetic profile as compared with corresponding amide-containing compounds.

### Method of Preparation

The method(s) by which the disclosed compounds can be prepared can vary, as will be apparent to one of ordinary skill in the art. In some embodiments, click chemistry reactions are used to synthesize compounds of the present disclosure, e.g., as reported in Keck, T. M. et al., Bioorg. Med. Chem. 2015, 23, 4000-4012, which is incorporated herein by reference in its entirety.

In some embodiments, a 1,2,3-triazole-containing compound as described herein can be prepared according to a method as generally depicted in Scheme 1, below.

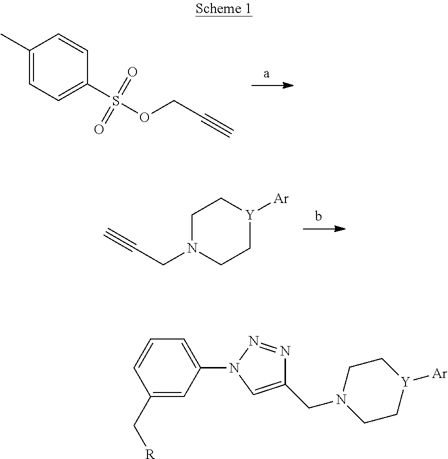

Representative reagents and conditions: (a) K₂CO₃, NaI, appropriate phenylpiperazine, acetone, reflux, 12 h; (b) (i) t-BuOH, H₂O, copper (II) sulfate pentahydrate, sodium ascorbate, appropriate acetylene, rt, 12 h.

In brief, as shown, commercially available tosylates were converted to acetylene-containing arylpiperazines or arylpiperidines by displacing the tosylate using the corresponding arylpiperazine or arylpiperidine amine under conditions. The acetylenes were coupled to the desired azides, formed in situ, to provide the desired triazole compounds. Thus, one suitable means for obtaining various compounds of the present disclosure involves a copper-catalyzed azide-alkyne cycloaddition click chemistry approach.

### Activity and Use of Disclosed Compounds

The compounds of the disclosure can find use, e.g., in treating or preventing diseases or disorders associated with dopamine binding, e.g., involving the central nervous system (CNS). In particular, compounds of Formula 1 (including 1a and 1b) provided above can bind as D₄R ligands (binding such dopamine receptors and thereby acting upon the CNS). As such, the present disclosure provides a method for treating or delaying the progression of disorders that are alleviated by binding to the dopamine (e.g., D₄R) receptors in a patient, the method comprising administering a therapeutically effective amount of at least one compound of Formula 1 (or Formula 1a or 1b) to the patient. In some embodiments, the compounds show selectivity for subtype D₄R binding over other dopamine receptor subtypes.

The disclosure may thus relate to the treatment of various conditions that may benefit from binding to the dopamine receptors. Although various such disorders and diseases are known, the compounds may be particularly applicable for treatment of cognitive disorders, treatment of addiction, e.g., cocaine addiction or opioid addiction, and treatment or prevention of drug addiction relapse (e.g., associated with cocaine or opioid addiction). In particular, the disclosure provides methods for treating cocaine or opioid addiction in animals, particularly humans and other mammals, and associated effects of these conditions.

The compounds may be further particularly applicable in treating L-DOPA-induced dyskinesias and impulse-control disorders, including substance abuse disorders (SUDs), eating disorders, and pathological gambling. In addition, the compounds may find use in treating cognitive deficits associated with neuropsychiatric disorders including schizophrenia and attention deficit hyperactivity disorder (ADHD). The compounds may, in some embodiments, reduce the adverse effects of opioid drugs like morphine.

The method of treatment generally includes administering a therapeutically effective amount of a compound of Formula 1 (or 1a or 1b), optionally in a pharmaceutical composition including one or more pharmaceutically acceptable carriers. The therapeutically effective amount is preferably sufficient to affect the D₄R subtype receptor. The therapeutically effective amount is further preferably sufficient to cause some relief to the patient in the symptoms of the disorder for which the patient is being treated. A therapeutically effective dosage amount of any specific formulation will vary somewhat from compound to compound, patient to patient, and may depend upon factors such as the condition of the patient and the route of delivery. When administered conjointly with other pharmaceutically active agents, even less of the compound of Formula 1 (or 1a or 1b) may be therapeutically effective. Furthermore, the therapeutically effective amount may vary depending on the specific condition to be treated.

The compounds provided herein can be administered once or several times a day. Dosages can be administered either by a single dose in the form of an individual dosage unit or several smaller dosage units or by multiple administration of subdivided dosages at certain intervals. Possible routes of delivery include buccally, subcutaneously, transdermally, intramuscularly, intravenously, orally, or by inhalation.

### Compositions

While it is possible for the compounds of Formula 1 (or Formulas 1A or 1B) to be administered in the raw chemical form, it is generally preferred for the compounds to be delivered as a pharmaceutical formulation. Accordingly, there are provided by the present disclosure pharmaceutical compositions comprising at least one compound capable of functioning as a partial agonist or as an antagonist of a dopamine (e.g., the D₄R) receptor. As such, formulations provided herein comprise a compound of Formula 1 (or 1A or 1B), as described above, or a pharmaceutically acceptable ester, amide, salt, or solvate thereof, together with one or more pharmaceutically acceptable carriers therefore, and optionally, other therapeutic ingredients.

By “pharmaceutically acceptable carrier” is intended a carrier that is conventionally used in the art to facilitate the storage, administration, and/or the healing effect of the agent. The carrier(s) must be pharmaceutically acceptable in the sense of being compatible with the other ingredients of the formulation and not unduly deleterious to the recipient thereof. A carrier may also reduce any undesirable side effects of the agent. Such carriers are known in the art. See, Wang et al. (1980) J. Parent. Drug Assn. 34(6):452-462, herein incorporated by reference in its entirety.

Adjuvants or accessory ingredients for use in the formulations of the present invention can include any pharmaceutical ingredient commonly deemed acceptable in the art, such as binders, fillers, lubricants, disintegrants, diluents, surfactants, stabilizers, preservatives, flavoring and coloring agents, and the like. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20” and “TWEEN 80”, and pluronics such as F68 and F88, available from BASF), sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). In some embodiments, the pharmaceutical compositions provided herein comprise one or more cyclodextrins.

Exemplary pharmaceutical excipients and/or additives suitable for use in the compositions according to the invention are listed in Remington: The Science & Practice of Pharmacy,” 21^{st} ed. Lippincott Williams & Wilkins (2006); in the Physician's Desk Reference, 64^{th} ed., Thomson PDR (2010); and in Handbook of Pharmaceutical Excipients, 6^{th} ed., Eds. Raymond C. Rowe et al., Pharmaceutical Press (2009), which are incorporated herein by reference.

Binders are generally used to facilitate cohesiveness of a tablet and ensure the tablet remains intact after compression. Suitable binders include, but are not limited to: starch, polysaccharides, gelatin, polyethylene glycol, propylene glycol, waxes, and natural and synthetic gums. Acceptable fillers include silicon dioxide, titanium dioxide, alumina, talc, kaolin, powdered cellulose, and microcrystalline cellulose, as well as soluble materials, such as mannitol, urea, sucrose, lactose, dextrose, sodium chloride, and sorbitol. Lubricants are useful for facilitating tablet manufacture and include vegetable oils, glycerin, magnesium stearate, calcium stearate, and stearic acid. Disintegrants, which are useful for facilitating disintegration of the tablet, generally include starches, clays, celluoses, algins, gums, and crosslinked polymers. Diluents, which are generally included to provide bulk to the tablet, may include dicalcium phosphate, calcium sulfate, lactose, cellulose, kaolin, mannitol, sodium chloride, dry starch, and powdered sugar. Surfactants suitable for use in the formulation according to the present invention may be anionic, cationic, amphoteric, or nonionic surface active agents. Stabilizers may be included in the formulations to inhibit or lessen reactions leading to decomposition of the active agent, such as oxidative reactions.

Formulations provided herein may include short-term, rapid-onset, rapid-offset, controlled release, sustained release, delayed release, and pulsatile release formulations, providing the formulations achieve administration of a compound as described herein. See Remington's Pharmaceutical Sciences (18^{th} ed.; Mack Publishing Company, Eaton, Pennsylvania, 1990), herein incorporated by reference in its entirety.

Pharmaceutical formulations according to the present disclosure are suitable for various modes of delivery, including oral, parenteral (including intravenous, intramuscular, subcutaneous, intradermal, and transdermal), topical (including dermal, buccal, and sublingual), and rectal administration. The most useful and/or beneficial mode of administration can vary, especially depending upon the condition of the recipient and the disorder being treated.

The pharmaceutical formulations may be conveniently made available in a unit dosage form, whereby such formulations may be prepared by any of the methods generally known in the pharmaceutical arts. Generally speaking, such methods of preparation comprise combining (by various methods) an active agent (e.g., a compound of Formula 1, 1A, or 1B as disclosed herein) or a pharmaceutically acceptable ester, amide, salt, or solvate thereof with a suitable carrier or other adjuvant, which may consist of one or more ingredients. The combination of the active ingredient with the one or more adjuvants is then physically treated to present the formulation in a suitable form for delivery (e.g., shaping into a tablet or forming an aqueous suspension).

Pharmaceutical formulations suitable as oral dosage may take various forms, such as tablets, capsules, caplets, and wafers (including rapidly dissolving or effervescing), each containing a predetermined amount of the active agent. The formulations may also be in the form of a powder or granules, a solution or suspension in an aqueous or non-aqueous liquid, and as a liquid emulsion (oil-in-water and water-in-oil). The active agent may also be delivered as a bolus, electuary, or paste. It is generally understood that methods of preparations of such dosage forms are generally known in the art, and any such method would be suitable for the preparation of the respective dosage forms for use in delivery of the compounds according to the present disclosure.

A tablet containing a compound as provided herein may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agent.

Solid dosage forms may be formulated so as to provide a delayed release of the active agent, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present invention may also be sustained release (i.e., releasing the active agent over a prolonged period of time), and may or may not also be delayed release. Sustained release formulations are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

Formulations for parenteral administration include aqueous and non-aqueous sterile injection solutions, which may further contain additional agents, such as anti-oxidants, buffers, bacteriostats, and solutes, which render the formulations isotonic with the blood of the intended recipient. The formulations may include aqueous and non-aqueous sterile suspensions, which contain suspending agents and thickening agents. Such formulations for parenteral administration may be presented in unit-dose or multi-dose containers, such as, for example, sealed ampoules and vials, and may be stores in a freeze-dried (lyophilized) condition requiring only the addition of the sterile liquid carrier, for example, water (for injection), immediately prior to use. Extemporaneous injection solutions and suspensions may be prepared from sterile powders, granules, and tablets of the kind previously described.

The compounds according to the present disclosure may also be administered transdermally, wherein the active agent is incorporated into a laminated structure (generally referred to as a “patch”) that is adapted to remain in intimate contact with the epidermis of the recipient for a prolonged period of time. Typically, such patches are available as single layer “drug-in-adhesive” patches or as multi-layer patches where the active agent is contained in a layer separate from the adhesive layer. Both types of patches also generally contain a backing layer and a liner that is removed prior to attachment to the skin of the recipient. Transdermal drug delivery patches may also be comprised of a reservoir underlying the backing layer that is separated from the skin of the recipient by a semi-permeable membrane and adhesive layer. Transdermal drug delivery may occur through passive diffusion or may be facilitated using electrotransport or iontophoresis.

Formulations for rectal delivery of the compounds of the present invention include rectal suppositories, creams, ointments, and liquids. Suppositories may be presented as the active agent in combination with a carrier generally known in the art, such as polyethylene glycol. Such dosage forms may be designed to disintegrate rapidly or over an extended period of time, and the time to complete disintegration can range from a short time, such as about 10 minutes, to an extended period of time, such as about 6 hours.

The compounds of Formulas 1, 1a, or 1b above may be formulated in compositions including those suitable for oral, buccal, rectal, topical, nasal, ophthalmic, or parenteral (including intraperitoneal, intravenous, subcutaneous, or intramuscular injection) administration. The compositions may conveniently be presented in unit dosage form and may be prepared by any of the methods well known in the art of pharmacy. All methods include the step of bringing a compound of the disclosed formulas into association with a carrier that constitutes one or more accessory ingredients. In general, the compositions are prepared by bringing a compound of the present disclosure into association with a liquid carrier to form a solution or a suspension, or alternatively, bringing a compound of the present disclosure into association with formulation components suitable for forming a solid, optionally a particulate product, and then, if warranted, shaping the product into a desired delivery form. Solid formulations provided herein, when particulate, will typically comprise particles with sizes ranging from about 1 nanometer to about 500 microns. In general, for solid formulations intended for intravenous administration, particles will typically range from about 1 nm to about 10 microns in diameter.

The amount of the compound of Formula 1 (or 1A or 1B) in the formulation will vary depending on the specific compound selected, dosage form, target patient population, and other considerations, and will be readily determined by one skilled in the art. The amount of the compound in the formulation will be that amount necessary to deliver a therapeutically effective amount of the compound to a patient in need thereof to achieve at least one of the therapeutic effects associated with the compounds of the disclosure. In practice, this will vary widely depending upon the particular compound, its activity, the severity of the condition to be treated, the patient population, the stability of the formulation, and the like. Compositions will generally contain anywhere from about 1% by weight to about 99% by weight of a compound of the invention, typically from about 5% to about 70% by weight, and more typically from about 10% to about 50% by weight, and will also depend upon the relative amounts of excipients/additives contained in the composition.

Aspects of the present disclosure are more fully illustrated with reference to the following examples. Before describing several exemplary embodiments of the technology, it is to be understood that the technology is not limited to the details of construction or process steps set forth in the following description. The technology is capable of other embodiments and of being practiced or being carried out in various ways. The following examples are set forth to illustrate certain aspects of the present technology and are not to be construed as limiting thereof.

### Experimentals

### Synthesis

Reaction conditions and yields were not optimized. Anhydrous solvents were purchased from Sigma Aldrich Corporation and were used without further purification. All other chemicals and reagents were purchased from Sigma-Aldrich Co. LLC, Aurora Fine Chemicals LLC, VWR Chemicals, Enamine, Acros Organics, and Alfa Aesar. All amine final products were converted into either oxalate or hydrochloride salt. Spectroscopic data and yields refer to the free base form of compounds. Flash chromatography was performed using silica gel (EMD Chemicals, Inc.; 230-400 mesh, 60 Å) by using Teledyne ISCO CombiFlash RF system. ¹H and ¹³C spectra were acquired using a JEOL ECZ-400S NMR spectrometer. All ¹H and ¹³C NMR experiments are reported in δ units and were measured relative to the signals for CDCl₃ (δ_{H} 7.26 ppm and δ_{C} 77.16 ppm), CD₂Cl₂ (δ_{H} 5.32 ppm and δ_{C} 53.84 ppm)(CD₃)₂CO (δ_{H} 2.05 ppm and δ_{C} 29.84 and 206.26 ppm) or CD₃OD (δ_{H} 3.31 ppm and δ_{C} 49.00 ppm). Chemical shifts, multiplicities, and coupling constants (J) have been reported and calculated using MNova 64. Combustion elemental analysis was performed by Atlantic Microlab, Inc. (Norcross, GA) and the results agree within ±0.4% of calculated values. c Log P values were calculated using ChemDraw version 20.0. Melting point determination was conducted using an SRS OptiMelt MPA100-Automated melting point apparatus and are uncorrected. Based on NMR and combustion elemental analysis data, all final compounds are ≥95% pure. Compounds 1-7 have been previously described in Keck, T. M. et al., J. Med. Chem. 2019, 62, 3722-3740, which is incorporated herein by reference in its entirety. General Method A. Propargyl p-toluenesulfonate (1 equiv.) and the specific piperazine (1 equiv.) were dissolved in acetone. Potassium carbonate (2 equiv.) and sodium iodide (5-10 mg) were added to the mixture. The reaction mixture was stirred at 60° C. overnight under N₂ atmosphere. After the reaction was complete, the solvent was removed under reduced pressure. The product was purified by flash chromatography (50% EtOAc:Hexanes) gradient to give the desired intermediates.

4-phenyl-1-(prop-2-yn-1-yl)piperidine (9). The compound was synthesized using propargyl p-toluenesulfonate (0.536 mL, 3.10 mmol), 4-phenylpiperidine (500 mg, 3.10 mmol), potassium carbonate (856.8 mg, 6.20 mmol) in acetone (12 mL) to yield dark orange solid. ¹H NMR (400 MHz, CDCl₃) δ 7.36-7.11 (m, 5H), 3.35 (dd, J=2.4, 0.8 Hz, 2H), 3.01 (dt, J=12.3, 3.2 Hz, 1H), 2.58-2.44 (m, 1H), 2.41-2.22 (m, 4H), 1.91-1.70 (m, 4H).

1-(prop-2-yn-1-yl)-4-(pyridin-2-yl)piperazine (10). The compound was synthesized using propargyl p-toluenesulfonate (1.06 mL, 6.13 mmol), 1-(2-pyridyl)piperazine (0.933 mL, 6.13 mmol), potassium carbonate (1.69 g, 12.25 mmol) in acetone (25 mL) to yield yellow solid. ₁H NMR (400 MHz, CDCl₃) δ 8.17 (ddd, J=5.0, 2.0, 0.9 Hz, 1H), 7.46 (ddd, J=8.6, 7.1, 2.0 Hz, 1H), 6.67-6.58 (m, 2H), 3.61-3.52 (m, 4H), 3.35 (d, J=2.5 Hz, 2H), 2.71-2.64 (m, 4H), 2.26 (t, J=2.4 Hz, 1H).

2-(4-(prop-2-yn-1-yl)piperazin-1-yl)pyrimidine (11). The compound was synthesized using propargyl p-toluenesulfonate (1.05 mL, 6.09 mmol), 2-(piperazin-1-yl)pyrimidine (0.86 mL, 6.09 mmol), potassium carbonate (1.68 g, 12.18 mmol) in acetone (25 mL). ¹H NMR (400 MHz, CD₂Cl₂) δ 8.28 (d, J=4.7 Hz, 2H), 6.47 (t, J=9.5, 4.7 Hz, 1H), 3.82 (dd, J=10.2, 5.0 Hz, 4H), 3.33 (d, J=2.5 Hz, 2H), 2.57 (dd, J=10.3, 5.1 Hz, 4H), 2.29 (t, J=2.5 Hz, 1H).

1-(5-chloropyridin-2-yl)-4-(prop-2-yn-1-yl)piperazine (12). The compound was synthesized using propargyl p-toluenesulfonate (0.88 mL, 5.06 mmol), 1-(5-chloropyridin-2-yl)piperazine (1.0 g, 5.06 mmol), potassium carbonate (1.66 g, 10.12 mmol) in acetone (25 mL). ¹H NMR (400 MHz, (CD₃)₂CO) δ 8.07 (dd, J=2.6, 0.7 Hz, 1H), 7.52 (ddd, J=9.0, 2.6, 0.7 Hz, 1H), 6.83 (d, J=9.0 Hz, 1H), 3.56 (dd, J=10.2, 5.1 Hz, 4H), 3.36 (d, J=2.5 Hz, 2H), 2.73 (t, J=2.4 Hz, 1H), 2.60 (dd, J=10.2, 5.1 Hz, 4H).

1-(naphthalen-1-yl)-4-(prop-2-yn-1-yl)piperazine (13). The compound was synthesized using propargyl p-toluenesulfonate (0.408 mL, 2.36 mmol), 1-(naphthalen-1-yl)-piperazine (500 mg, 2.36 mmol), potassium carbonate (650.92 mg, 4.71 mmol) in acetone (12 mL) to yield light yellow solid. ¹H NMR (400 MHz, CDCl₃) δ 8.23-8.16 (m, 1H), 7.84-7.77 (m, 1H), 7.54 (d, J=8.1 Hz, 1H), 7.50-7.42 (m, 2H), 7.39 (dd, J=8.2, 7.4 Hz, 1H), 7.09 (dd, J=7.4, 1.1 Hz, 1H), 3.43 (d, J=2.5 Hz, 2H), 3.18 (s, 4H), 2.87 (s, 4H), 2.32 (t, J=2.4 Hz, 1H).

General Method B. The specific 1-azidobenzene (1 equiv.) and the intermediate (1.0 equiv.) were dissolved in a water/tert-butanol mixture. Sodium ascorbate (0.1 equiv.) and copper (II) sulfate pentahydrate (0.01 equiv.) were individually dissolved in H₂O and added to the solution. The heterogeneous mixture was stirred at room temperature overnight under N₂ atmosphere. After the reaction was complete, the solvent was removed under reduced pressure. The product was subjected to flash column chromatography to provide the desired compounds. All final products were converted into oxalate salts.

4-phenyl-1-((1-(m-tolyl)-1H-1,2,3-triazol-4-yl)methyl)piperidine (14). The compound was synthesized using 1-azido-3-methylbenzene (222.2 mg, 1.51 mmol), 4-phenyl-1-(prop-2-yn-1-yl)piperidine (9) (1.51 mmol, 200 mg), sodium ascorbate (30 mg, 0.15 mmol), copper (II) sulfate pentahydrate (3.7 mg, 0.015 mmol) in a mixture of tert-Butanol (0.3 g) and H₂O (8 mL). The product was purified by flash column chromatography (60% EtOAc/Hexanes) to yield an orange solid (61%). ¹H NMR (400 MHz, CDCl₃) δ 7.97 (s, 1H), 7.64-7.60 (m, 1H), 7.55 (dd, J=8.0, 2.2 Hz, 1H), 7.42 (t, J=7.8 Hz, 1H), 7.32-7.22 (m, 5H), 7.18 (ddt, J=7.3, 5.7, 1.3 Hz, 1H), 3.75 (s, 2H), 3.09 (d, J=11.8 Hz, 2H), 2.53 (tt, J=11.5, 4.5 Hz, 1H), 2.46 (s, 3H), 2.21 (td, J=11.4, 3.2 Hz, 2H), 1.81 (qd, J=12.6, 3.6 Hz, 4H). ¹³C NMR (101 MHz, CD₂Cl₂) δ 146.94, 146.03, 140.46, 137.54, 129.82, 129.58 (2C), 128.70 (2C), 127.20, 126.39, 121.29, 121.21, 117.69, 56.03, 54.46, 53.96, 42.79, 33.89 (2C), 21.52. The oxalate salt was precipitated from 2-propanol. MP: 216.5-217.2° C. Anal. (C₂₁H₂₄N₄·C₂H₂O₄) C, H, N.

1-(pyridin-2-yl)-4-((1-(m-tolyl)-1H-1,2,3-triazol-4-yl)methyl)piperazine (15). The compound was synthesized using 1-azido-3-methylbenzene (500 mg, 3.755 mmol), 1-(prop-2-yn-1-yl)-4-(pyridin-2-yl)piperazine (10) (750 mg, 3.755 mmol), sodium ascorbate (75 mg, 0.3755 mmol), copper (II) sulfate pentahydrate (10 mg, 0.03755 mmol) in a mixture of tert-Butanol (0.5 g) and H₂O (12 mL). The product was purified by flash column chromatography (95% EtOAc/Hexanes) to yield a clay-colored crude product (69%). ¹H NMR (400 MHz, CDCl₃) δ 8.13 (t, J=2.3 Hz, 1H), 7.98 (d, J=8.1 Hz, 1H), 7.59 (d, J=7.0 Hz, 1H), 7.55-7.36 (m, 3H), 7.26 (t, J=7.6 Hz, 1H), 6.64 (t, J=8.8 Hz, 1H), 6.58 (ddd, J=8.5, 5.5, 3.1 Hz, 1H), 3.77 (d, J=7.7 Hz, 2H), 3.53 (dq, J=8.5, 4.6 Hz, 4H), 2.68-2.61 (m, 4H), 2.44 (d, J=7.2 Hz, 3H). ¹³C NMR (101 MHz, CD₂Cl₂) δ 159.90, 148.21, 148.19, 145.34, 140.51, 137.66, 129.85, 129.67, 121.44, 121.35, 117.76, 113.47, 107.25, 53.65, 53.12 (2C), 45.42 (2C), 21.53. The Oxalate salt was precipitated from 2-propanol. MP: 227.3-228.1° C. Anal. (C₁₉H₂₂N₆·C₂H₂O₄) C, H, N.

2-(4-((1-(m-tolyl)-1H-1,2,3-triazol-4-yl)methyl)piperazin-1-yl)pyrimidine (16) The compound was synthesized using 1-azido-3-methylbenzene (463 mg, 3.46 mmol), 2-(4-(prop-2-yn-1-yl)piperazin-1-yl)pyrimidine (11) (700 mg, 3.46 mmol), sodium ascorbate (68.5 mg, 0.346 mmol), copper (II) sulfate pentahydrate (8.64 mg, 0.0346 mmol) in a mixture of tert-Butanol (0.5 g) and H₂O (10 mL). The product was purified by flash column chromatography (90% EtOAc/Hexanes) to yield a red solid (75%). ¹H NMR (400 MHz, CDCl₃) δ 8.27 (t, J=3.7 Hz, 2H), 7.97 (d, J=2.7 Hz, 1H), 7.58 (s, 1H), 7.52 (d, J=8.3 Hz, 1H), 7.40 (td, J=7.9, 2.5 Hz, 1H), 7.25 (d, J=7.6 Hz, 1H), 6.46 (q, J=4.0 Hz, 1H), 3.80 (p, J=4.8 Hz, 4H), 3.75 (d, J=2.5 Hz, 2H), 2.57 (t, J=4.8 Hz, 4H), 2.44 (s, 3H). ¹³C NMR (101 MHz, CD₂Cl₂) δ 162.10, 157.98 (2C), 145.38, 140.48, 140.47, 137.44, 129.82, 129.63, 121.37, 121.29, 117.69, 110.14, 53.57 (2C), 43.91 (2C), 21.51. The oxalate salt was precipitated from 2-propanol. MP: 224-224.5° C. Anal. (C₁₈H₂₁N₇·C₂H₂O₄) C, H, N.

1-(5-chloropyridin-2-yl)-4-((1-(m-tolyl)-1H-1,2,3-triazol-4-yl)methyl)piperazine (17) The compound was synthesized using 1-azido-3-methylbenzene (398 mg, 2.97 mmol), 1-(5-chloropyridin-2-yl)-4-(prop-2-yn-1-yl)piperazine (12) (700 mg, 2.97 mmol), sodium ascorbate (55.27 mg, 0.297 mmol), copper (II) sulfate pentahydrate (7.49 mg, 0.0297 mmol) in a mixture of tert-Butanol (0.5 g) and H₂O (10 mL). The product was purified by flash column chromatography (80% EtOAc/Hexanes) to yield a white solid (57%). ¹H NMR (400 MHz, CDCl₃) δ 8.07 (d, J=2.6 Hz, 1H), 7.96 (s, 1H), 7.59 (s, 1H), 7.52 (d, J=8.3 Hz, 1H), 7.46-7.37 (m, 2H), 7.27 (d, J=7.6 Hz, 1H), 6.60 (d, J=9.1 Hz, 1H), 3.77 (s, 2H), 3.52 (t, J=5.1 Hz, 4H), 2.62 (t, J=5.0 Hz, 4H), 2.45 (s, 3H). ¹³C NMR (101 MHz, CD₂Cl₂) δ 158.16, 146.36, 145.27, 140.46, 137.40, 137.26, 129.81, 129.63, 121.37, 121.26, 120.10, 117.66, 108.03, 53.54, 52.89 (2C), 45.46 (2C), 21.50. The oxalate salt was precipitated from 2-propanol. MP: 222.8-223.1° C. Anal. (C₁₉H₂₁ClN₆·C₂H₂O₄) C, H, N.

1-(naphthalen-1-yl)-4-((1-(m-tolyl)-4,5-dihydro-1H-1,2,3-triazol-4-yl)methyl)piperazine (18). The compound was synthesized using 1-azido-3-methylbenzene (212.98 mg, 1.59 mmol), 1-(naphthalen-1-yl)-4-(prop-2-yn-1-yl)piperazine (13) (1.59 mmol, 400 mg), sodium ascorbate (31.5 mg, 0.159 mmol), copper (II) sulfate pentahydrate (4.0 mg, 0.0159 mmol) in a mixture of tert-Butanol (0.5 g) and H₂O (8 mL). The product was purified by flash column chromatography (70% EtOAc/Hexanes) to yield an orange/brown solid (53%). ¹H NMR (400 MHz, CDCl₃) δ 8.23-8.18 (m, 1H), 8.02 (d, J=1.8 Hz, 1H), 7.85-7.80 (m, 1H), 7.63 (d, J=2.2 Hz, 1H), 7.55 (dd, J=8.4, 3.8 Hz, 2H), 7.52-7.45 (m, 2H), 7.45-7.37 (m, 2H), 7.27 (d, J=7.6 Hz, 1H), 7.11 (d, J=7.4 Hz, 1H), 3.88 (d, J=1.7 Hz, 2H), 3.16 (s, 4H), 2.87 (s, 4H), 2.46 (s, 3H). ¹³C NMR (101 MHz, CD₂Cl₂) δ 150.04, 145.41, 140.51, 137.53, 135.13, 129.85, 129.66, 129.19, 128.65, 126.23, 126.14, 125.63, 123.98, 123.66, 121.46, 121.35, 117.75, 114.97, 53.74, 53.62 (2C), 53.30 (2C), 21.54. The oxalate salt was precipitated from 2-propanol. MP: 184.1-184.9° C. (C₂₄H₂₅N₅·C₂H₂O₄) C, H, N.

1-((1-(3-ethylphenyl)-1H-1,2,3-triazol-4-yl)methyl)-4-(pyridin-2-yl)piperazine (19). The compound was synthesized using 1-azido-3-ethylbenzene (441.54 mg, 3 mmol), 1-(prop-2-yn-1-yl)-4-(pyridin-2-yl)piperazine (10) (603.81 mg, 3 mmol), sodium ascorbate (59.43 mg, 0.3 mmol), copper (II) sulfate pentahydrate (7.5 mg, 0.03 mmol) in a mixture of tert-Butanol (0.5 g) and H₂O (10 mL). The product was purified by flash column chromatography (80% EtOAc/Hexanes) to yield a transparent solid (49%). ¹H NMR (400 MHz, CDCl₃) δ 8.14-8.10 (m, 1H), 7.98 (s, 1H), 7.60 (s, 1H), 7.53 (d, J=8.1 Hz, 1H), 7.48-7.40 (m, 2H), 7.28 (d, J=7.6 Hz, 1H), 6.63 (d, J=8.6 Hz, 1H), 6.58 (dd, J=7.1, 4.9 Hz, 1H), 3.76 (s, 2H), 3.52 (t, J=5.1 Hz, 4H), 2.74 (q, J=7.6 Hz, 2H), 2.63 (t, J=5.1 Hz, 4H), 1.27 (t, J=7.6 Hz, 3H). ¹³C NMR (101 MHz, CD₂Cl₂) δ 159.89, 148.18, 146.82, 145.41, 137.64, 137.56, 129.92, 128.52, 121.42, 120.25, 117.98, 113.43, 107.22, 53.65, 53.11 (2C), 45.41 (2C), 29.10, 15.60. The oxalate salt was precipitated from 2-propanol. MP: 212.1-212.7° C. (C₂₀H₂₄N₆·C₂H₂O₄) C, H, N.

### Radioligand Binding Assays

Binding at dopamine D₂-like receptors was determined similarly to previously described methods and identical to the methods previously used in Keck, T. M. et al., J. Med. Chem. 2019, 62, 3722-3740, which is incorporated herein by reference in its entirety.

Membranes were prepared from HEK293 cells stably expressing human D_{2L}R, D₃R, or D₄R grown in a 50:50 mix of DMEM and Ham's F12 culture media, supplemented with 20 mM HEPES, 2 mM L-glutamine, 0.1 mM non-essential amino acids, 1× antibiotic/antimycotic, 10% heat-inactivated fetal bovine serum, and 200 μg/mL hygromycin (Life Technologies, Grand Island, NY) and kept in an incubator at 37° C. and 5% CO₂. Upon reaching 80-90% confluence, cells were harvested using pre-mixed Earle's Balanced Salt Solution (EBSS) with 5 mM EDTA (Life Technologies) and centrifuged at 3,000 rpm for 10 min at 21° C. The supernatant was removed, and the pellet was resuspended in 10 mL hypotonic lysis buffer (5 mM MgCl₂·6 H₂O, 5 mM Tris, pH 7.4 at 4° C.) and centrifuged at 14,500 rpm (˜25,000 g) for 30 min at 4° C. The pellet was then resuspended in fresh EBSS binding buffer made from 8.7 g/L Earle's Balanced Salts without phenol red (US Biological, Salem, MA), 2.2 g/L sodium bicarbonate, pH to 7.4. A Bradford protein assay (Bio-Rad, Hercules, CA) was used to determine the protein concentration and membranes were diluted to 500 g/mL and stored in a −80° C. freezer for later use.

Radioligand competition binding experiments were conducted using freshly dissolved drugs on each test day. Each test compound was diluted into 10 half-log serial dilutions using 30% DMSO vehicle, ranging from 100 μM to 0.3 nM final concentrations, adjusted depending on compound solubility and to optimize binding curve calculations. Previously frozen membranes were thawed and diluted in fresh EBSS binding buffer to 200 μg/mL (for hD_{2L}R or hD₃R) or 400 μg/mL (for hD₄R) for binding. Radioligand competition reactions were conducted in 96-well plates containing 300 μl fresh EBSS binding buffer, 50 μl of diluted test compound, 100 μl of diluted membranes (20 μg/well total protein for hD_{2L}R and hD₃R, or 40 μg/well total protein for hD₄R), and 50 μl of [³H]N-methylspiperone radioligand diluted in binding buffer (0.4 nM final concentration; Perkin Elmer). Nonspecific binding was determined using 10 μM (+)-butaclamol (Sigma-Aldrich, St. Louis, MO) and total binding was determined with 30% DMSO vehicle. All compound dilutions were tested in triplicate and the reaction incubated for 1 hour at RT. The reaction was terminated by filtration through Perkin Elmer Uni-Filter-96 GF/B plates, presoaked for 1 hour in 0.5% polyethylenimine, using a Brandel 96-Well Plates Harvester Manifold (Brandel Instruments, Gaithersburg, MD). The filters were washed (3×1 mL/well) with ice-cold binding buffer. After drying overnight at RT, Perkin Elmer MicroScint 20 Scintillation Cocktail (45 μL) was added to each well and filters were counted using a Perkin Elmer MicroBeta2 scintillation counter. IC₅₀ values for each compound at each receptor were determined from dose-response curves and Kᵢ values were calculated using the Cheng-Prusoff equation When a complete inhibition couldn't be achieved at the highest tested concentrations, Kᵢ values have been extrapolated by constraining the bottom of the dose-response curves (=0% residual specific binding) in the non-linear regression analysis. These analyses were performed using GraphPad Prism versions 6.00-8.00 (GraphPad Software, San Diego, CA). All results were rounded to three significant figures. Kᵢ values were determined from at least 3 independent experiments and are reported as means±SEM.

The receptor binding data in HEK293 cells and c Log P values (and data for corresponding amide-linkers in place of the 1,2,3-triazole linkers) are provided in Table 2, below.

TABLE 2
Human Dopamine D₂-like Receptor Binding Data in HEK293 cells for ligands with amide (comparative) or 1,2,3-triazole moieties^{a}

| | | | Kᵢ (nM) ± SEM | | | | |
|---|---|---|---|---|---|---|---|
| Compound | | | [³H]N-methlyspiperone | | | Receptor Selectivity | |
| No. | Structure | cLogP | D₂R | D₃R | D₄R | D₂R/D₄R | D₃R/D₄R |
| 1 (Comparative) | 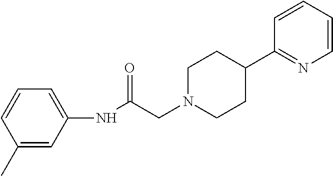 [SMILES: Cc1cccc(NC(=O)CN2CCC(c3ccccn3)CC2)c1] | 2.92 | 6250 ± 380 | 1680 ± 450 | 54.2 ± 7.0 | 115 | 31 |
| 5 (Comparative) | 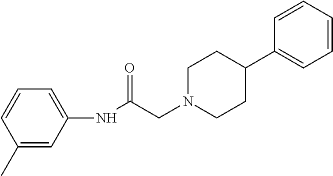 [SMILES: Cc1cccc(NC(=O)CN2CCC(c3ccccc3)CC2)c1] | 4.41 | 821± 35 | 433 ± 137 | 25.8 ± 9.0 | 32 | 17 |
| 14 | 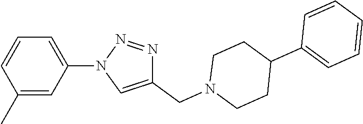 [SMILES: Cc1cccc(-n2cc(CN3CCC(c4ccccc4)CC3)nn2)c1] | 4.62 | 410 ± 121 | 25,800 ± 21,400 | 21.3 ± 10.0 | 19 | 1212 |
| 2 (Comparative) | 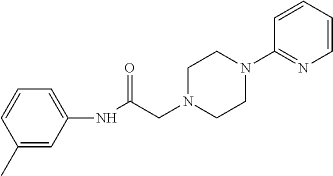 [SMILES: Cc1cccc(NC(=O)CN2CCN(c3ccccn3)CC2)c1] | 2.84 | >10,000 | >10,000 | 212 ± 63 | >47 | >47 |
| 15 | 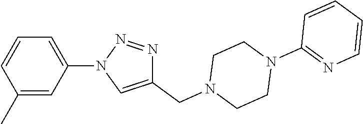 [SMILES: Cc1cccc(-n2cc(CN3CCN(c4ccccn4)CC3)nn2)c1] | 2.98 | 11,400 ± 800 | 35,800 ± 6500 | 16.2 ± 0.6 | 704 | 2210 |
| 4 (Comparative) | 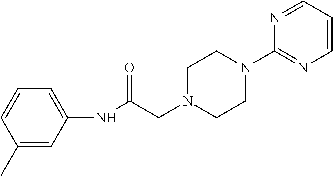 [SMILES: Cc1cccc(NC(=O)CN2CCN(c3ncccn3)CC2)c1] | 2.07 | 6400 ± 3800 | >10,000 | 318 ± 95 | 20 | >31 |
| 16 | 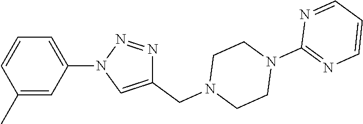 [SMILES: Cc1cccc(-n2cc(CN3CCN(c4ncccn4)CC3)nn2)c1] | 2.21 | 67,900 ± 31,200 | 91,800 ± 56,000 | 42.2 ± 9.8 | 1610 | 2176 |
| 7 (Comparative) | 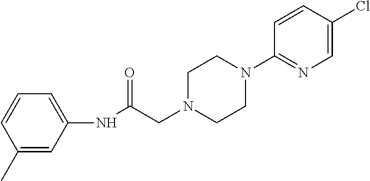 [SMILES: Cc1cccc(NC(=O)CN2CCN(c3ccc(Cl)cn3)CC2)c1] | 3.63 | >50,000 | >50,000 | 95.0 ± 26.0 | >526 | >526 |
| 17 | 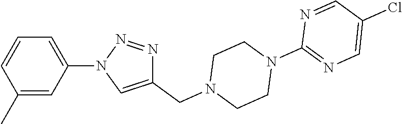 [SMILES: Cc1cccc(-n2cc(CN3CCN(c4ncc(Cl)cn4)CC3)nn2)c1] | 3.78 | >100,000 | >100,000 | 77.7 ± 19.9 | >1287 | >1287 |
| 6 (Comparative) | 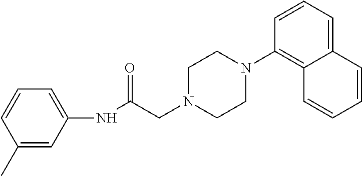 [SMILES: Cc1cccc(NC(=O)CN2CCN(c3cccc4ccccc34)CC2)c1] | 4.96 | 1490 ± 100 | 11,500 ± 3000 | 28.4 ± 8.0 | 52 | 402 |
| 18 | 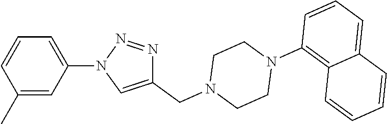 [SMILES: Cc1cccc(-n2cc(CN3CCN(c4cccc5ccccc45)CC3)nn2)c1] | 5.10 | 6540 ± 5370 | 10,800 ± 8200 | 4.33 ± 1.02 | 1510 | 2504 |
| 3 (Comparative) | 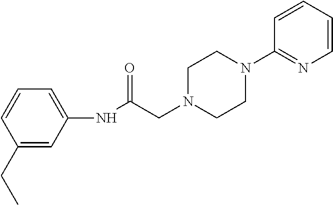 [SMILES: CCc1cccc(NC(=O)CN2CCN(c3ccccn3)CC2)c1] | 3.36 | 6250 ± 380 | 1680 ± 450 | 54.2 ± 7.0 | 115 | 31 |
| 19 | 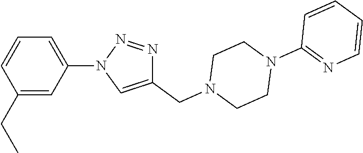 [SMILES: CCc1cccc(-n2cc(CN3CCN(c4ccccn4)CC3)nn2)c1] | 3.51 | 821 ± 35 | 433 ± 137 | 25.8 ± 9.0 | 32 | 17 |

^{a}Kᵢ values determined by competitive inhibition of [³H]N-methylspiperone binding in membranes harvested from HEK293 cells stably expressing hD₂R, hD₃R, or hD₄R. All Kᵢ values are presented as means ± SEM. ^{†}Data previously reported in Keck, T.M. et al., J. Med. Chem. 2019, 62, 3722-3740, which is incorporated herein by reference in its entirety.

As outlined above, this study was designed to determine the ability of each analog to displace the radioligand [³H]N-methylspiperone and affinity was determined using the Cheng-Prusoff equation as described in further detail above. In addition, c Log P values were calculated to provide measures of polarity. Overall, as shown in Table 2, the majority of the compounds exhibited c Log P values of less than 5 and new triazole library members consistently demonstrated higher binding affinity for D₄R over D₂R and D₃R.

Comparing the binding affinities across each pair of amide and triazole analogs, a majority of the triazole analogs had comparable affinity for the D₄R than their amide analogs (Table 2), indicating that the substitution is well-tolerated compared to previously published binding results as disclosed in Keck, T. M. et al., J. Med. Chem. 2019, 62, 3722-3740, which has been incorporated herein by reference in its entirety. Compound 14 maintained binding affinity for D₄R (21.3 nM) comparable to its analog 5 (25.8 nM), with 19-fold and 1,212-fold selectivity over D₂R and D₃R, respectively. 15 displayed higher binding affinity for D₄R (16.2 nM) compared to its analog 2 (212 nM), resulting in improved 704-fold and 2,210-fold selectivity over D₂R and D₃R, respectively. 16 displayed higher binding affinity for D₄R (42.2 nM) compared to its analog 4 (318 nM), resulting in improved 1,610-fold and 2,176-fold selectivity over D₂R and D₃R, respectively. Compound 17 displayed higher binding affinity for D₄R (77.7 nM) comparable to its analog 7 (95.0 nM), with >1,287-fold selectivity over D₂R and D₃R. Compound 18 displayed higher binding affinity for D₄R (4.33 nM) comparable to its analog 6 (28.4 nM), with 1,510-fold and 2,504-fold selectivity over D₂R and D₃R, respectively. Compound 19 displayed higher binding affinity for D₄R (19.7 nM) comparable to its analog 3 (67.9 nM), with 2,389-fold and >5,076-fold selectivity over D₂R and D₃R, respectively. While the triazole substitution typically resulted in modestly favorable affinity gains at D₄R, a conservative evaluation of these results indicates that the triazole substitution shows no negative impact on D₄R affinity or subtype selectivity as compared with the amide analogues.

### Functional Assays

### β-Arrestin Recruitment Assay

The effects of the triazole linker on β-arrestin recruitment to the D₂-like receptors was evaluated. Functional analyses of each compound were completed using the DiscoverX β-arrestin recruitment assay (Table 2). Analogs were tested in both agonist and antagonist modes using Chinese hamster ovary (CHO) cells stably expressing a prolink-tagged D₂R, D₃R, or D₄R and a β-arrestin2 tagged with the remaining portion of β-galactosidase in an enzyme complementation assay.

Assays were conducted in a manner identical to the methods previously used in Keck & Free et al. (2019), previously incorporated herein by reference, using the DiscoverX PathHunter technology (Eurofins DiscoverX, Fremont, CA). Briefly, CHO-K1 cells stably expressing the human D₂R long isoform, D₃R, or D₄R (Eurofins DiscoverX) were maintained in Ham's F12 media supplemented with 10% fetal bovine serum, 100 U/mL penicillin, 100 μg/ml streptomycin, 800 μg/ml G418 and 300 μg/ml hygromycin at 37° C., 5% CO₂, and 90% humidity. The cells were seeded in 7.5 μl media at a density of 2,625 cells/well in 384-well black, clear-bottom plates. The following day, the compounds were diluted in PBS with 0.2 mM sodium metabisulfite. The cells were treated with 16 concentrations of a compound in triplicate and incubated at 37° C. for 90 minutes. Tropix Gal-Screen Substrate (Applied Biosystems, MA) was diluted in Gal-Screen buffer A (Applied Biosystems) 1:25 and added to cells according to the manufacturer's recommendations followed by a 30-45-minute incubation at room temperature in the dark. Luminescence was measured on a Hamamatsu FDSS μCell reader.

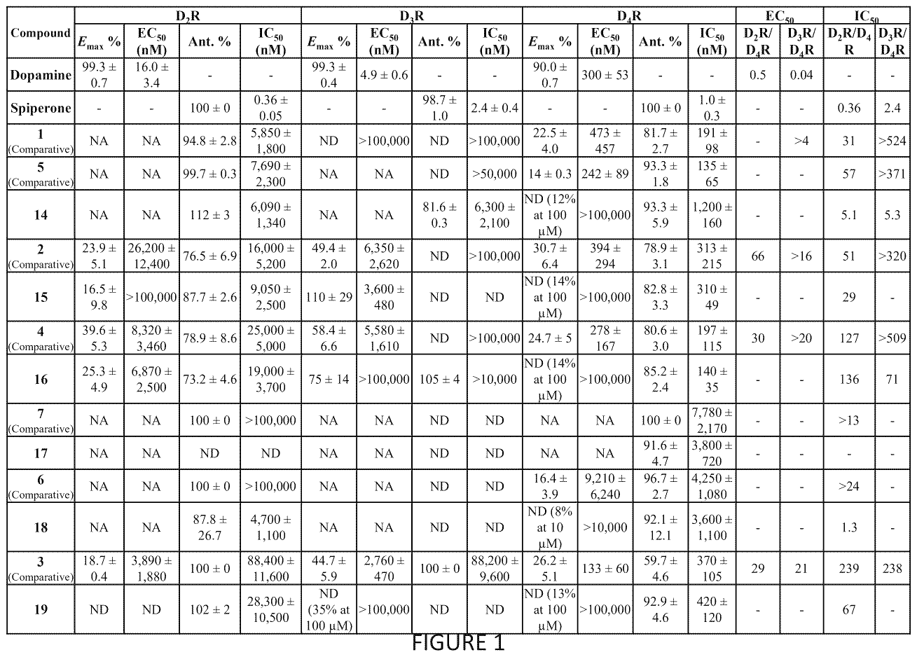

Data was collected in triplicate and transferred to GraphPad Prism 9, where it was fit with non-linear regression curve fit equations. The data were normalized to the percent maximum dopamine response (agonist mode) or the EC₈₀ of dopamine (antagonist mode). The Hill coefficients of the concentration-response curves did not significantly differ from unity with the data fitting to a single site model. Data in the table provided in FIG. 1 are from at least three independent replicates. The data from each experiment was fit as described above with the Eₘₐₓ, Ant. %, EC₅₀, and IC₅₀ values extracted from the non-linear regression. The Eₘₐₓ, Ant. %, EC₅₀ and IC₅₀ values were meaned together using descriptive statistics in Prism and reported as mean±SEM.

FIG. 1 provides a table of the D₂R-, D₃R-, and D₄R-mediated β-arrestin recruitment. Dopamine was used as a control in all agonist mode assays. Spiperone was included in all antagonist mode assays for the D₂R and D₄R. ND: Not Determined due to an incomplete curve. Compounds were tested alone (agonist mode) and with an EC₈₀ concentration of dopamine (antagonist mode) for their ability to alter β-arrestin recruitment to D₂R, D₃R, and D₄R. Fold selectivity for the D₄R over the D₂R and D₃R were also calculated and presented in the table of FIG. 1. Efficacy/antagonist % (Ant. %) values were obtained from nonlinear regression of meaned data obtained from at least three independent experiments with triplicate measures. Values are presented as means±SEM. Data for the Comparative examples provided in the table were previously reported in Keck, T. M. et al., J. Med. Chem. 2019, 62, 3722-3740, previously incorporated herein by reference.

In agonist mode, Eₘₐₓ values for each compound are in comparison to dopamine while the antagonist mode assays were normalized to spiperone. In general, the triazole analogs displayed potencies and efficacies consistent with their respective amide analogs. The triazole substitutions did not affect the potencies of the compounds for the D₂-like receptors with a few exceptions detailed below. At the D₄R, the triazole 14 was less potent (1,200 nM) than the amide analog 5 (135 nM) but the efficacy was not affected (93%). The efficacies indicated they were antagonists but had very low potency (>6,000 nM) at the D₂R and D₃R. The triazole 16 did not show partial agonist activity at the D₄R while the amide 4 analog had 25% efficacy and 278 nM potency for recruiting β-arrestin. There was a similar effect with 6 and 18 as well as 3 and 19 pairs of amide vs triazole. Both amides show low partial agonist activity while the triazole analogs did not. All the D₂R, D₃R, and D₄R β-arrestin recruitment results are shown in the table of FIG. 1 and indicate that the triazole substitution with the amide was well-tolerated and was not detrimental for β-arrestin recruitment antagonism with the exception of 14.

Taken together, the binding and functional results indicate that the triazole linker was well-tolerated and was even favorable for many of the analogs tested. Compound 18 was an especially interesting analog due to its high binding affinity at the D₄R and complete lack of functional activity at the D₃R. Additionally, 18 had very low binding affinity and potency for antagonizing β-arrestin recruitment to the D₂R adding further evidence that 18 is highly D₄R selective over the homologous D₂R and D₃R.

### Molecular Modeling and Docking

### Model and Ligand Preparation:

To initiate the molecular dynamics (MD), the initial crystal structure was obtained from the RCSB PDB website. The crystal structure obtained was PDB ID 5WIU (see Wang, S. et al., Science 2017, 358, 381-386 and Li, Y. et al., Proceedings—2019 IEEE International Conference on Bioinformatics and Biomedicine 2019, 303-310, which are incorporated herein by reference), which has a resolution of 2.6 Å. The T4-lysozyme that is used to take the place of the intracellular loop 3 was deleted and replaced with N-methyl and acetyl caps on the termini of this deleted region. All molecules in the PDB were deleted except for the receptor. The protonation states of ionizable residues were assigned by the H++ server (see Gordon, J. C. et al., Nucleic Acids Res. 2005, 33, W368-W371, incorporated herein by reference in its entirety), with pH 7.4.

L-dopamine was used as the ligand for the initial MD simulation. The model for L-dopamine was generated using the 2D sketcher function of Schrodinger's Maestro, the Ligprep protocol was used to generate conformations of this ligand and protonation states using a pH of 7.4+/−2.0. See Johnston, R. C. et al., J. Chem. Theory Comput. 2023, 19, 2380-2388, which is incorporated herein by reference in its entirety. Twenty-five generated molecules were requested and only one conformation generated the positively charged amine, which was kept.

The model was placed into Schrodinger's Maestro for visualization, followed by their protein preparation protocol, and finally receptor grid generation as part of their docking protocol. The receptor grid was formed using residue D115^{3.32} as the center. Dopamine was docked into the D₄R. The highest scoring pose was kept which resembles the binding mode of dopamine in literature.

The receptor was given to Packmol-memgen (Schott-Verdugo, S.; Gohlke, H. PACKMOL-Memgen: A Simple-To-Use, Generalized Workflow for Membrane-Protein-Lipid-Bilayer System Building. J. Chem. Inf. Model 2019, 59, 2522-2528, incorporated herein by reference) to create a lipid membrane for the simulation. Lipids were generated in a 9:1 ratio of POPC:CHL1, respectively. Additional ions to mimic a salt concentration of 150 mm NaCl were added by packmol-memgen. Antechamber (Wang, J.; Wang, W.; Kollman, P. A.; Case, D. A. Automatic atom type and bond type perception in molecular mechanical calculations. J. Mol. Graph. Model 2006, 25, 247-260, incorporated herein by reference) was used to assign a +1 charge to L-dopamine. The tleap module was used to prepare the system. See Case, D. A. et al., The Amber biomolecular simulation programs. J. Comput. Chem. 2005, 26, 1668-1688, incorporated herien by reference in its entirety. Tleap used the Amber FF19SB forcefield (Tian, C. et al., J. Chem. Theory Comput. 2020, 16, 528-552, incorporated herein by reference) for the protein, OPC water model (Izadi, S. et al., J. Phys. Chem. Lett. 2014, 5, 3863-3871, incorporated herein by reference), gaff2 (Wang, J. et al., J. Comput. Chem. 2004, 25, 1157-1174, incorporated herein by reference) for the ligand, and lipid21 (Dickson, C. J. et al., J. Chem. Theory Comput. 2022, 18, 1726-1736, incorporated herein by refernece) for the membrane. Parmed (Shirts, M. R. et al., J. Comput. Aided Mol. Des. 2017, 31, 147-161, incorporated herein by reference) was utilized to activate hydrogen mass repartitioning (HMR) which allows for a 4 femtosecond (fs) timestep.

### Molecular Dynamics Simulation:

MD simulations were performed using the AMBER suite (Case, D. A. et al., J. Comput. Chem. 2005, 26, 1668-1688, incorporated herein by reference). The model underwent five minimization steps. First, the model underwent 5000 cycles of steep descent, followed by 5000 cycles of conjugate gradient with a restraint weight of 25 kcal·mol⁻¹. Å⁻² on the membrane and protein. Next, the model underwent 5000 cycles of steep descent, followed by 5000 cycles of conjugate gradient with a restraint weight of 5 kcal·mol⁻¹. Å⁻² on the membrane and protein. In the third minimization step, the model underwent 5000 cycles of steep descent, followed by 5000 cycles of conjugate gradient with a restraint weight of 5 kcal·mol⁻¹. Å⁻² on the protein. In the fourth minimization step, the model underwent 5000 cycles of steep descent, followed by 5000 cycles of conjugate gradient with a restraint weight of 1 kcal·mol⁻¹. Å⁻² on the protein. In the fifth minimization step, the model underwent 5000 cycles of steep descent, followed by 10000 cycles of conjugate gradient with no restraints.

The SHAKE algorithm was applied to all bonds connected to hydrogen atoms with a time step of 4 fs. The system was heated from 0 K to 100 K in 5 picoseconds (ps) with restraints of 5 kcal·mol⁻¹. Å⁻² on the membrane and protein. The Langevin thermostat (Loncharich, R. J. et al., Biopolymers 1992, 32, 523-535; Berendsen, H. J. C. et al., J. Chem. Phys. 1984, 81, 3684-3690; and Åqvist, J. et al., Chem. Phys. Lett. 2004, 384, 288-294, which are incorporated herein by reference) was used with a collision frequency value of 2.0 ps and cutoff of 10.0 Å. The system then underwent additional heating to 310K over 100 ps with restraints still held. The Berendsen barostat^{47b} was used during the equilibration process which occurred in three steps. First, restraints of 5 kcal·mol⁻¹. Å⁻² were placed on the ligand and the protein backbone for 2 nanoseconds (ns). Next, restraints of 5 kcal·mol⁻¹. Å⁻² were placed on the ligand and the alpha carbons of the protein for 2 nanoseconds. Lastly, all atoms were allowed to move freely for 100 ns prior to the production run. The Monte Carlo barostat^{47c} was then used for the production run with a target pressure of 1 atm. The production ran for 2.5 microseconds (μs).

### Docking Studies:

After 2.5 μs of MD simulations, frames of the trajectory were manually visualized to obtain a frame with the extended binding pocket (EBP) visible. A frame from the first 100 nanoseconds was used for docking purposes. The model was placed into Schrodinger's Maestro for visualization, followed by their protein preparation protocol, and finally receptor grid generation as part of their docking protocol. The receptor grid was formed using residue D115^{3.32} as the center. The amide-based and triazole-based compounds were drawn using the 2D sketcher functionality and converted to 3D structures. The compounds underwent Maestro's LigPrep protocol using a pH of 7.4+/−2.0 (Johnston, R. C. et al., J. Chem. Theory Comput. 2023, 19, 2380-2388, incorporated herein by reference) and was asked to generate twenty conformers. Four conformations of each compound were generated with only one containing the positively charged amine so one of the four was kept while the others were discarded. This conformation was used for the Schrodinger Glide SP Protocol (Friesner, R. A. et al., J. Med. Chem. 2004, 47, 1739-1749, incorporated herein by reference) and docked into the D4 receptor. With fifty poses requested, fifteen were generated.

DeepAtom Binding Energy Analysis: The compounds coinciding with the highest reported docking score were converted to pdbqt files using Obabel (O'Boyle, N. M.; Banck, M.; James, C. A.; Morley, C.; Vandermeersch, T.; Hutchison, G. R. Open Babel: An open chemical toolbox. J. Cheminform. 2011, 3, 33, incorporated herein by reference). After this, DeepAtom (Li, Y. et al., Proceedings—2019 IEEE International Conference on Bioinformatics and Biomedicine 2019, 303-310, incorporated herein by reference) was used to predict the binding energies of the compounds.

Based on the above protocols, Comparative compounds 2-7 and Compounds 14-19 were evaluated by in silico studies. Overall, a modest but consistent improvement in D₄R affinity was found in the 1,2,3,-triazole analogs compared to their amide counterparts. To determine the mechanisms of these improvements, molecular dynamics (MD) simulations of the structure of the human D₄R in complex with nemonapride (PDB ID: 5WIU) in complex with L-dopamine were used to create a model of D₄R in an agonist-bound state. Following MD simulations, compounds 2-7 and 14-19 were docked into the receptor orthosteric site. Models with the highest docking score for each receptor-ligand pair were then analyzed using DeepAtom as described above to predict the binding energies of each compound (Table 3). Table 3 displays calculated binding energy scores using DeepAtom, where the left columns of the table represent amide compounds, with matching triazole-based analogs in the right columns.

TABLE 3
Deep Atom Binding affinity scores for
compounds 2-7 and 14-19 at the D₄R.

| | |
|---|---|
| Deep Atom Binding affinity | Deep Atom Binding affinity |
| scores for amide analogs | scores for triazole analogs |

| Compound | Score | Compound | Score |
|---|---|---|---|
| Number | (kcal/mol) | Number | (kcal/mol) |
| 5A ^{a} | −10.16 | 14A ^{a} | −10.45 |
| 5B ^{a} | −10.10 | 14B ^{a} | −10.59 |
| 2 | −9.61 | 15 | −10.48 |
| 4 | −9.63 | 16 | −10.49 |
| 7 | −9.63 | 17 | −10.38 |
| 6 | −9.01 | 18 | −10.19 |
| 3 | −9.56 | 19 | −10.45 |

^{a} Compounds 5A and 5B represent probable alternative docking pose conformations of amide 5. Similarly, compounds 14A and 14B represent probable alternative docking pose conformations of triazole analog 14. The A conformations represent the “opposite pose”, and the B conformation represent the “consistent pose” (i.e., conformationally consistent with the docking of 2-7 and 14-19).

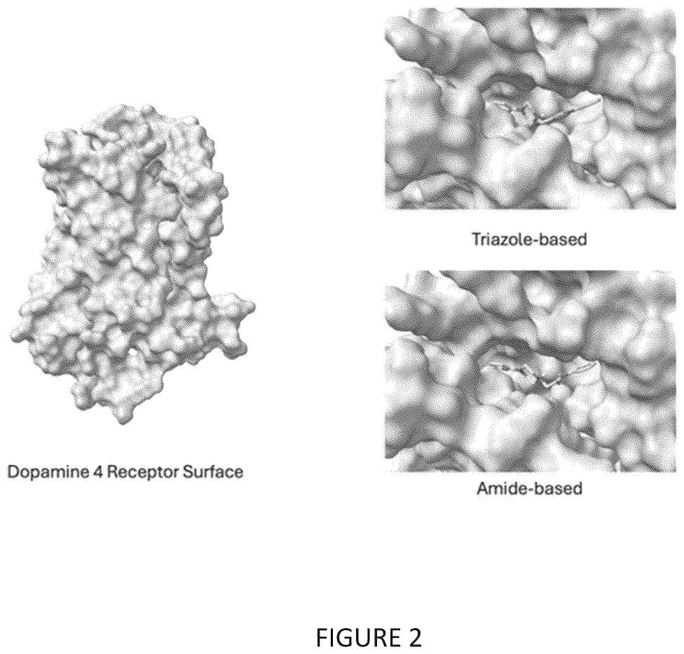

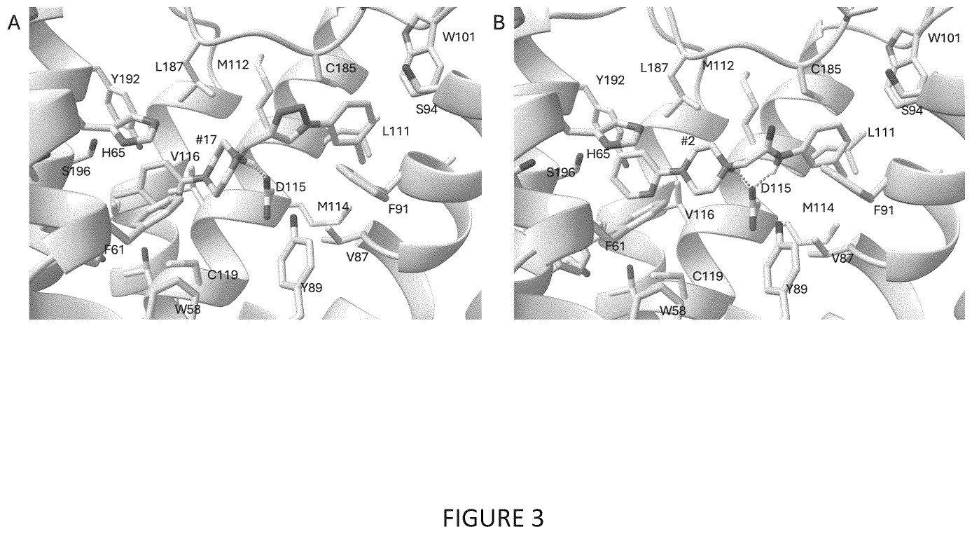

After docking, fifteen poses were generated for each compound. FIG. 2 shows the surface of the D₄R from afar and a zoom-in of the binding site, composed of the orthosteric and extended-binding pocket (EBP) sites. All ligands showed consistency in binding mode and orientation, however analogs 5 and 14 each also showed a matching variant “opposite pose” described in more detail below. A representative set of amides and triazole compounds was chosen (Comparative compound 2 and compound 15, respectively) to illustrate comparative binding interactions. FIG. 3 illustrates the interactions of 2 and 17 with the amino acid side chains found in the binding site.

The poses seen consistently amongst all compounds, exemplified by amide 2 and triazole 17, share key features. The methyl phenyl group of these compounds prefer placement into the EBP, which is formed through W101. It appears that this pocket cannot hold large aromatic or hydrophobic moieties. In FIG. 3(A), D115 displayed a salt bridge with the quaternary amine of 17, a conserved interaction amongst dopamine receptor binders. Compound 2 shows the same salt bridge formation, but the amide nitrogen provides an additional interaction in the form of a hydrogen bond with D115.

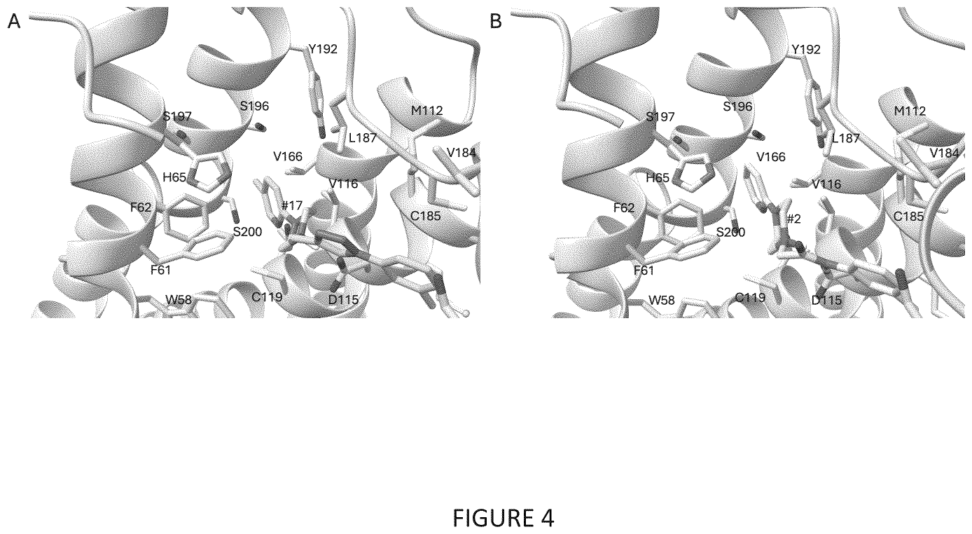

FIG. 4 displays interactions within the orthosteric binding pocket (OBP) for 17 and 2. In this pocket, hydrophobic interactions dominate. Normally with dopamine, the hydroxyls of the catechol would interact with S196/197, however, these compounds do not have this ability and thus will not form those interactions. Pi-pi interactions can be seen through the ring and F61/62, with slight aromatic interactions of H₆₅ and hydrophobic interactions from V116/166, L187, and C119.

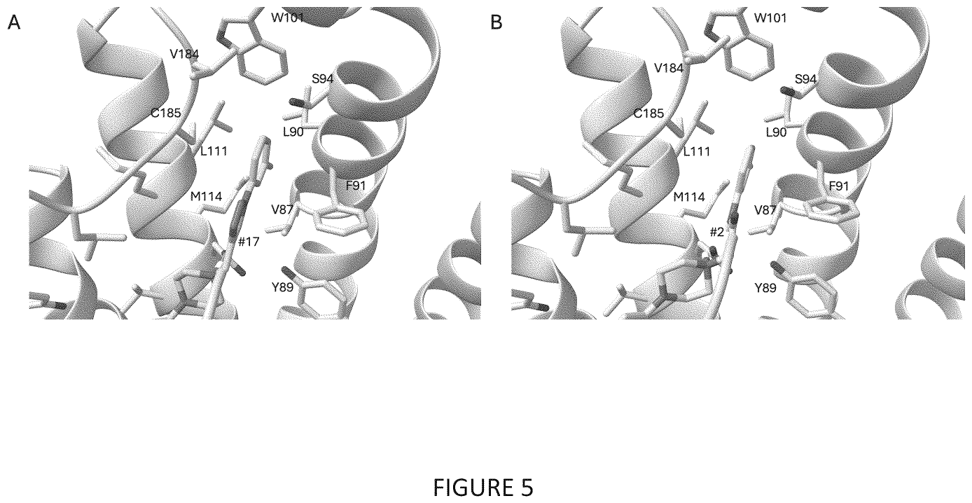

FIG. 5 shows the compounds forming interactions within the extended-binding pocket (EBP). Hydrophobic and pi-pi interactions also dominate here. The compounds form hydrophobic interactions with M114, V87, L90, L111, and V184. A nearby F91 could be used for potential pi-pi interactions with the triazole-based compounds through the triazole ring.

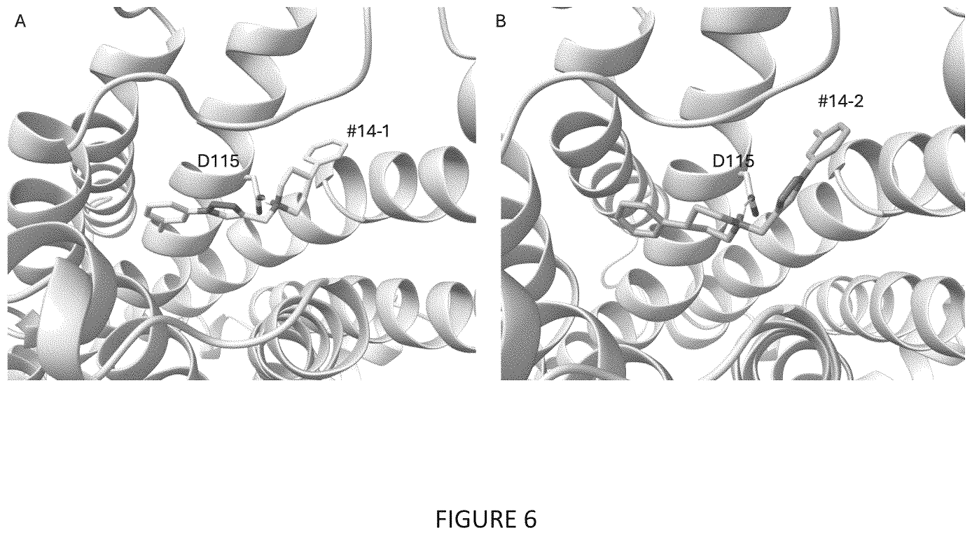

As mentioned previously, compounds 5 and 14 showed two plausible orientations while docking to D₄R, a the “consistent pose” (i.e., conformationally consistent with the docking of 2-7 and 14-19) that maintains the interactions described above, as well as an “opposite pose” with a flipped orientation. The Maestro docking functionality gave equivalent docking scores to the “opposite pose” and “consistent pose” orientations. After using DeepAtom, both pose orientations are produce a similar binding energy (Table 3). FIG. 6 displays triazole-based compound 14 in the “opposite pose” (panel A; 14A in Table 3) and “consistent pose” (panel B; 14B in Table 3). Surprisingly, it appears that the phenyl ring on compounds 5 and 14 can be equally accommodated by either side of the binding site.

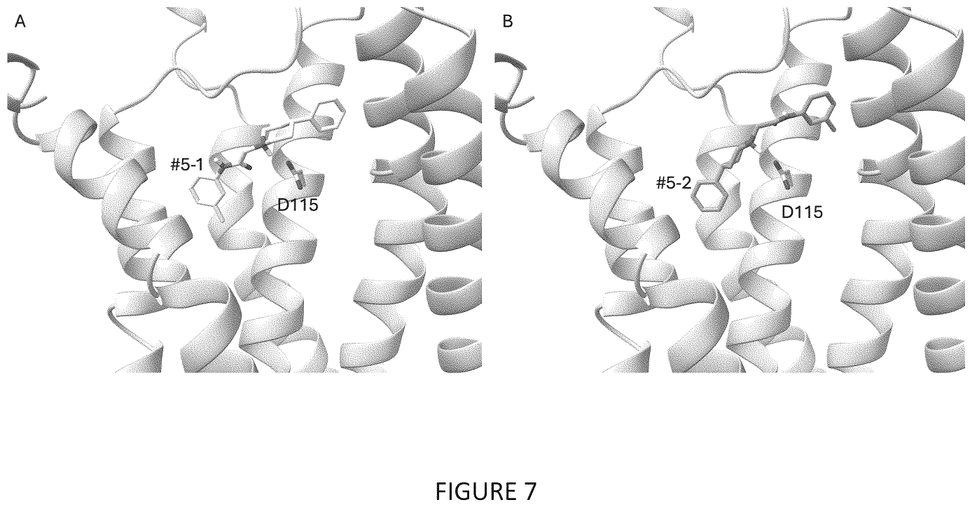

FIG. 7 displays amide-based compound 5 in the “opposite pose” (panel A; 5A in Table 3) and “consistent pose” (panel B; 5B in Table 3). In these images the amide nitrogen is no longer participating in H-bonding with the conserved D115; it is not readily apparent why no docking poses for 5 showed this interaction while all other compounds did. As with the alternate poses for compound 14, although not intending to be limited by theory, it is possible that the similarly sized aromatic rings on each end of the ligand can be accommodated by either end of the binding site.

Considering the overall docking results, the “consistent pose” was strongly preferred when the aromatic ring of 5 or 14 (a methylphenyl) features moieties that create a more electron-deficient ring, such as pyridines or chlorine. The possibility of sterics being a player here may contribute to equal favoring of either pose. The accommodability of the binding site could also be impacted by the use of a select number of frames during the MD simulations performed—it may be possible that throughout the trajectory, one pose may be preferred over the other. Although not intending to be limited by theory, the contribution of electron-withdrawing groups and electron-donating groups may additionally play a role within the orthosteric site. There are more aromatic moieties in the OBP compared to the EBP.

All docking poses underwent binding affinity calculations using DeepAtom, a 3D-convolutional neural network used to calculate binding affinities with high accuracy (Table 2). Overall, the triazole-based compounds produced a moderately more negative binding energy compared to the amide-based compounds. This is consistent with the modest improvement in D₄R affinity seen in the radioligand binding studies presented above.

### Rat and Human Microsomal Stability Assays

Phase I metabolic stability assays were conducted using rat and human liver microsomes as previously described (see Battiti, F. O. et al., J. Med. Chem. 2019, 62, 6287-6314, incorporated herein by reference), with minor modifications. In brief, the reactions were carried out with 100 mM potassium phosphate buffer, pH 7.4, in the presence of NADPH regenerating system (1.3 mM NADPH, 3.3 mM glucose 6-phosphate, 3.3 mM MgCl₂, 0.4 U/mL glucose-6-phosphate dehydrogenase, 50 μM sodium citrate). Negative controls without cofactors were assessed to determine the non-CYP-mediated metabolism. Compound disappearance was monitored over time using a liquid chromatography and tandem mass spectrometry (LC/MS) method. All reactions were performed in triplicate.

Chromatographic analysis was performed on a Dionex ultra high-performance LC system coupled with Q Exactive Focus orbitrap mass spectrometer (Thermo Fisher Scientific Inc., Waltham MA). Separation was achieved using Agilent Eclipse Plus column (100×2.1 mm i.d.; maintained at 35° C.) packed with a 1.8 μm C18 stationary phase. The mobile phase used was composed of 0.1% Formic Acid in Acetonitrile and 0.1% Formic Acid in water with gradient elution, starting with 2.5% organic phase (from 0 to 2 min) linearly increasing to 99% (from 2 to 5.5 min), and re-equilibrating to 2.5% by 6.5 min. The total run time for each analyte was 6.5 min. Pumps were operated at a flow rate of 0.3 mL/min. The mass spectrometer controlled by Xcalibur software 4.0.27.13 (Thermo Scientific) was operated with a HESI ion source in positive ionization mode. Compounds were identified in the full-scan mode (from m/z 50 to 750) by comparing t=0 samples with t=30 min and t=60 min samples.

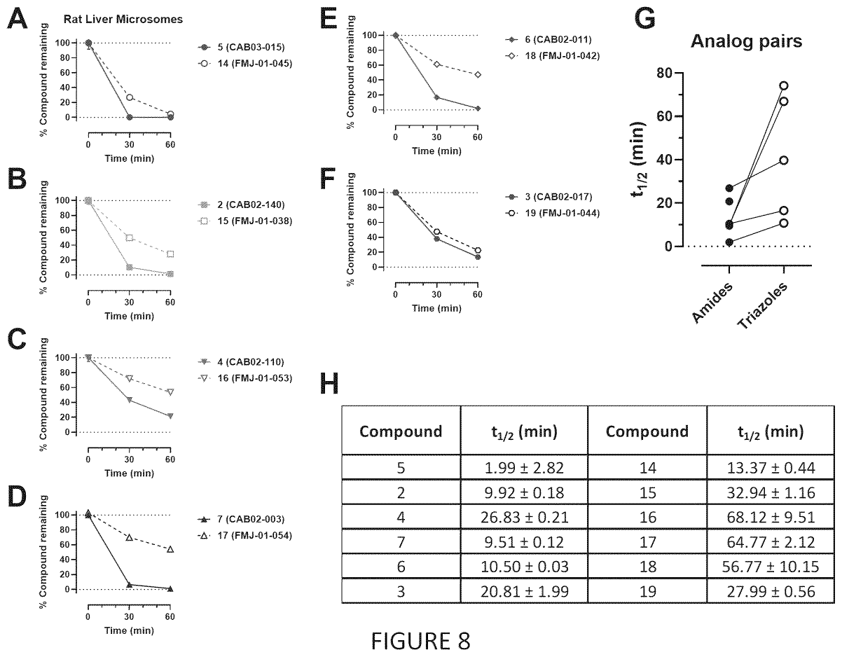

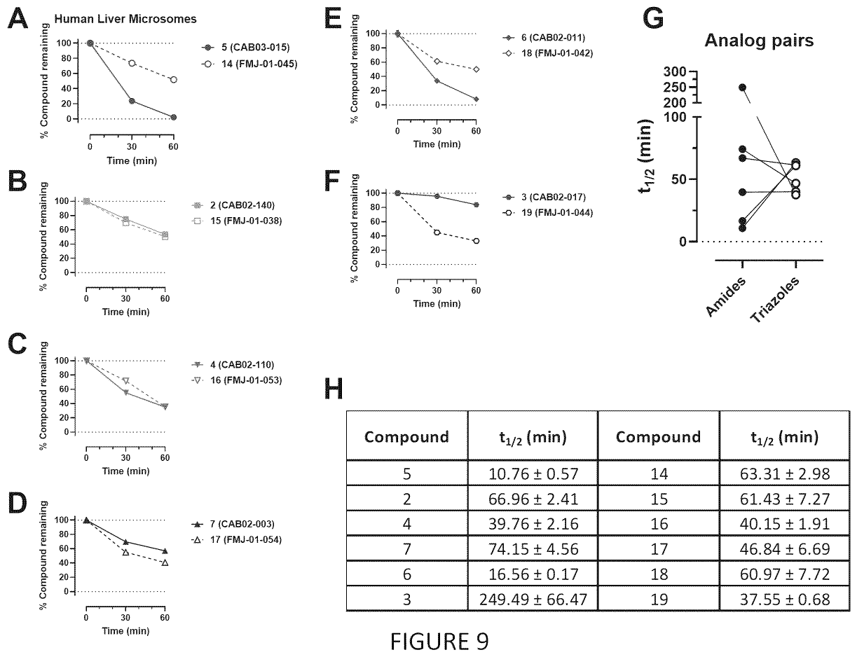

FIGS. 8 and 9 provide results of the Phase I metabolic stability evaluation of compounds 2-7 and 14-19 using rat and human liver microsomes. Incubation of compounds 2-7 and 14-19 with rat (FIG. 8) and human (FIG. 9) liver microsomes in the presence of NADPH resulted in time-dependent degradation. Overall, these results clearly indicate that amides 2-7 have lower metabolic stability compared to matching triazoles 14-19 in rat liver microsomes. Considering the main goal of this study was to identify a mechanism to improve compound stability in rats for further behavioral studies, this proved to be a successful substitution. Amides 2-7 had greater overall stability in human liver microsomes, and the triazole substitution resulted in a mix of improved, unchanged, and reduced microsomal half-life calculations, ranging from approximately 37-64 minutes, which are still suitable for continued development.

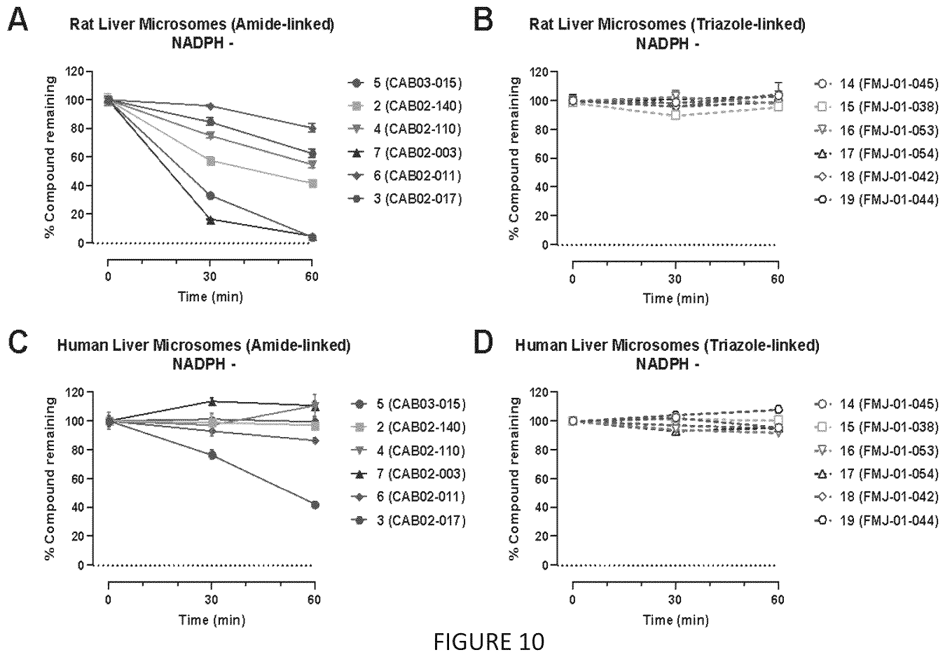

The non-Phase I metabolic stability of compounds 2-7 and 14-19 using rat and human liver microsomes is demonstrated in FIG. 10. Incubation of compounds 2-7 and 14-19 with rat (FIGS. 10A-B) and human (FIGS. 10C-D) liver microsomes in the absence of NADPH generally resulted in time-dependent compound degradation at a much slower rate than in the presence of NADPH. Notably, several amide compounds (2-7) have considerable microsomal instability—particularly in rat microsomal studies—even in the absence of the NADPH cofactor necessary for cytochrome P450-mediated metabolism. This may represent metabolism by hydrolases that can specifically attack the amide. Evidence in support of this hypothesis is shown by the remarkable stability of all triazole analogs (14-19) in the absence of NADPH in FIGS. 10B and D.

### Pharmacokinetics Study in Rats:

Pharmacokinetic studies in Sprague Dawley (SD) rats were conducted according to protocols approved by the Animal Care and Use Committee at Johns Hopkins University. SD rats obtained from Harlan were maintained on a 12 h light-dark cycle with ad libitum access to food and water. Test compound was administered via i.p. injection at a dose of 10 mg/kg (100% saline vehicle, 10 ml/kg volume). The rats were sacrificed at specified time points (0.25, 0.5 h, 1, 2, 4, and 6 h) post drug administration. For the collection of plasma and brain tissue, animals were euthanized with CO₂, and blood samples were collected in heparinized microtubes by cardiac puncture. Brains were dissected and immediately flash-frozen (−80° C.). Blood samples were spun at 2000 g for 15 min, and plasma was removed and stored at −80° C. until analysis (as described below).

### Bioanalysis:

Quantitation of 18 was performed using liquid chromatography with tandem mass spectrometry (LC/MS-MS) methods. Briefly, calibration standards were prepared using respective tissue (naïve plasma and brain) with additions of the test compound. For quantifying the test compound in the pharmacokinetic samples, plasma samples (20 μL) were processed using a single liquid extraction method by addition of 100 μL of acetonitrile containing internal standard (losartan: 0.5 μM), followed by vortex-mixing for 30 s and then centrifugation at 10,000×g for 10 min at 4° C. Brain tissues were diluted 1:5 w/v with acetonitrile containing losartan (0.5 μm) and homogenized, followed by vortex-mixing and centrifugation at 10,000×g for 10 min at 4° C. A 50 μL aliquot of the supernatant was diluted with 50 μL of water and transferred to 250 μL polypropylene autosampler vials sealed with Teflon caps. 2 μL of the sample was injected into the LC/MS/MS system for analysis. Chromatographic analysis was performed using an Accela ultra high-performance system consisting of an analytical pump and an autosampler coupled with a TSQ Vantage mass spectrometer. Separation of analyte was achieved at ambient temperature using Agilent Eclipse Plus column (100× 2.1 mm i.d.) packed with a 1.8 μm C18 stationary phase. The mobile phase consisted of 0.1% formic acid in acetonitrile and 0.1% formic acid in water with gradient elution, starting with 10% organic phase (from 0 to 1 min) linearly increasing to 95% (from 1 to 2 min), and re-equilibrating to 10% by 3 min. The total run time for each analyte was 3.5 min. Pumps were operated at a flow rate of 0.4 mL/min. The [M+H]⁺ ion transition of test compound 18 (m/z 384.2→144.1, 182.1, 225.1) and losartan (IS) (m/z 423.2→207.1, 377.2) were used. Plasma concentrations (nmol/ml) as well as brain tissue concentrations (nmol/g) were determined and plots of mean plasma concentration versus time were constructed. Non-compartmental analysis modules in Phoenix WinNonlin version 7.0 (Certara USA, Inc., Princeton, NJ) were used to quantify exposures (AUC₀₋ₜ) and half-life (t_{1/2}).

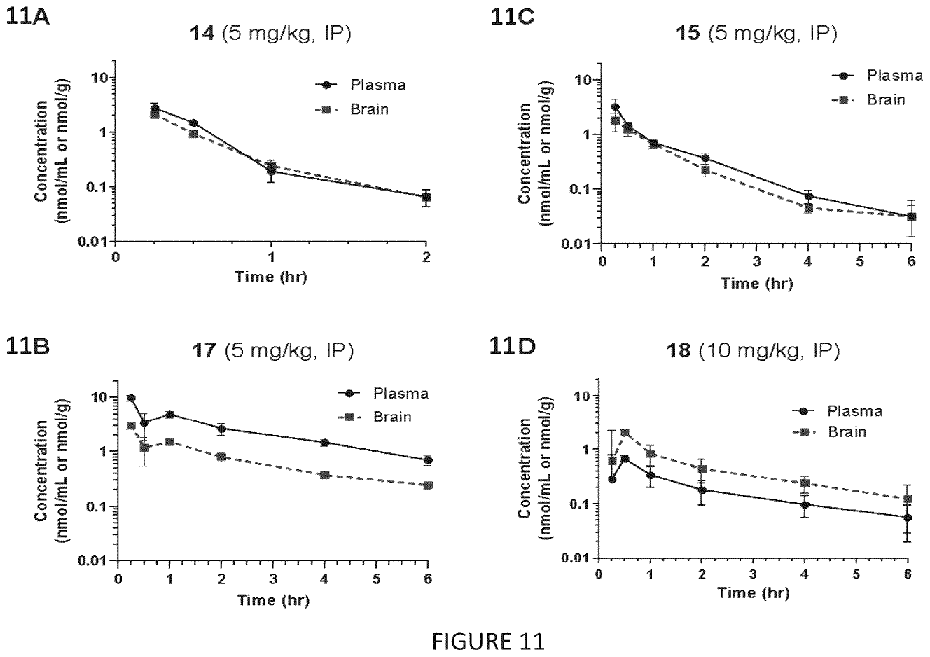

FIGS. 11A and 11B provide the results of the in vivo pharmacokinetic profile studies on compound 18 (selected due to its adequate stability profile) in rats. Sprague Dawley rats were dosed with 18 (10 mg/kg, i.p.) and plasma and brain levels of were measured 0-6 hours post-dose. The results from the pharmacokinetic analysis are shown in FIG. 11A-B. Compound 18 demonstrated good exposure in both plasma and brain, with AUC₀₋ₜ values of 1.36 nmol·h/mL and 3.57 nmol·h/g, respectively. Compound 18 was observed to have an excellent brain penetration index (AUC_{brain/plasma} ratio) of 2.7 and with an apparent half-life of ˜1 hour (t_{1/2}). The detailed pharmacokinetic parameters of 18 are provided in FIG. 11B.

Overall, evidence from prior studies indicates that D₄R signaling may play important roles in cognition and attention, but major questions remain about how D₄R signaling contributes to various neuropsychiatric disorders or the physiological consequences associated with the polymorphic nature of the human DRD4 gene. Pharmacological targeting of D₄Rs may be useful for treating cognitive deficits associated with neuropsychiatric disorders including schizophrenia and ADHD. D₄R agonism has been explored as a strategy to reduce the adverse effects of opioid drugs like morphine. D₄R antagonism may have potential to treat L-DOPA-induced dyskinesias and impulse-control disorders, including SUDs, eating disorders, and pathological gambling. The importance of targeting D₄Rs in treating these complex pathologies, especially in regard to the extent of receptor activation or inhibition, remains unknown, partially due to a lack of suitable compounds for investigating these pathways.

In this study, the bioisosteric replacement of amide linkers with a 1,2,3-triazole moiety resulted in modest improvements in D₄R affinity when compared to their parent (amide-containing) compounds, with minimal changes or modest improvements in D₂-like subtype selectivity and signaling profiles. Molecular modeling studies support the idea that the 1,2,3-triazole substitution minimally impacted ligand orientation in the binding site, with small improvements in binding energies consistent with improved D₄R affinity in radioligand competition binding studies. 1,2,3-triazole analogs provided substantive gains in metabolic stability compared to matching amides, particularly in rat microsomal studies. Notably, the triazole substitution appears to have completely eliminated non-Phase I (NADPH-) metabolism of these compounds.

Full characterization of triazole analog 18 indicates it is a highly D₄R-selective antagonist and a viable lead compound for behavioral testing. 18 demonstrated improved metabolic stability in human and rat liver microsomes in comparison to its amide analog, with in vivo plasma half-life and brain penetration values that are acceptable for rat behavioral studies. Overall, this new 1,2,3-triazole analog library represents compounds with high D₄R affinity, good selectivity over D₂R and D₃R, and a range of efficacy profiles.

### Attention-Deficit-Hyperactivity Disorder (ADHD) Studies in Rats:

Adolescent male SHR/NCrl and Wistar rats (4 weeks old at study onset) were obtained from Charles River Laboratories (Wilmington, MA, USA) and pair-housed (except during delay discounting testing) under controlled environmental conditions (12:12 h light/dark cycle; lights on at 6:00 a.m.; temperature 22±2° C.) with ad libitum access to food and water except during behavioral experiments. Rats were allowed to acclimate to the housing environment for one week before testing. All procedures were conducted in accordance with the Public Health Service Policy on Humane Care and Use of Laboratory Animals and the Guide for the Care and Use of Laboratory Animals and were approved by the Loma Linda University Institutional Animal Care and Use Committee (IACUC #24-015). All drug compounds were dissolved in 20-30% DMSO and 0.9% saline to achieve the desired concentrations. Two cohorts of SHR/NCrl and Wistar rats were used in this study. The first cohort underwent a series of behavioral assessments, namely the open field test, novel object preference test, and Y-maze task, with 24-hour intervals between each test to minimize potential carryover effects. The second cohort, which was subjected to food restriction and task-specific training, was used exclusively for delay discounting task. The effects of vehicle (control) or drug treatments were assessed during the behavioral tests as described below. All experiments were conducted by an investigator blinded to the treatment conditions. Between sessions, the behavioral apparatuses were thoroughly cleaned with Quatricide to eliminate residual odors and prevent cross-contamination.

The open field test is a well-established method for assessing spontaneous locomotor behavior in rodents. The procedures of the open field tests followed previously reported protocols (dela Peña et al., 2021) with some modifications. Briefly, after intraperitoneal (i.p.) injection with either vehicle, FMJ-01-38 (Compound 15), or FMJ-01-54 (Compound 17), each rat was placed individually and immediately in an activity chamber (47×47×50 cm), and locomotion was recorded for 30 minutes using an automated tracking system (Ethovision System, Noldus I. R., Wageningen, The Netherlands). Prior work from our group demonstrated that locomotor activity in SHR/NCrl rats peaked within the first 30 minutes and subsequently declined (dela Peña et al., 2021), supporting the selection of this time window for assessing drug-induced changes in activity. Total distance traveled (in centimeters) and movement duration (in seconds) were quantified to evaluate drug effects on locomotor activity of animals.

The novel object preference test was used to evaluate attention and recognition memory, behavioral domains commonly altered in ADHD animal models. Testing was conducted in a Plexiglas arena (47×47×50 cm) equipped with an overhead camera and spanned three consecutive days: habituation, familiarization, and novel object testing. On Day 1, rats were allowed to freely explore the empty arena for 10 minutes. On Day 2, two identical red cone-shaped objects were placed in the arena, and rats were allowed to explore for 10 minutes. On Day 3, one familiar object was replaced with a novel object differing in both shape and color. Drug or vehicle treatments were administered 20 minutes before the novel object testing session, which lasted 5 minutes. Exploration time, defined as sniffing or touching the object within 2 cm, was recorded manually by an experimenter blinded to treatment conditions. The percent preference for the novel object was calculated as [time exploring novel/(time exploring novel+time exploring familiar)]×100. A reduced preference for the novel object was interpreted as impaired attention or recognition memory.

Spontaneous alternation behavior in the Y-maze task reflects both attentional and working memory processes. Deficits in attention are manifested as a reduced percentage of spontaneous alternation. The Y-maze procedure followed methods described previously, e.g., in de la Peña et al., Behavioural Brain Research, 291, 268-276 (2015) and de la Peña et al., European Journal of Pharmacology, 892, 173826 (2021), which are incorporated herein by reference in their entireties. Rats received intraperitoneal injections of vehicle or test drug 20 minutes before testing. Each animal was placed at the end of one arm of the maze and allowed to explore freely for 8 minutes. Arm entries were recorded manually, with an entry defined as placement of all four paws and the tail within an arm. From these data, the number of actual alternations (successive entries into all three arms in overlapping triplets) and the maximum possible alternations (total arm entries−2) were determined using methods as outlined in the dela Peña references incorporated by reference herein above. The percentage of spontaneous alternation, calculated as (actual/maximum alternations)×100, served as the primary index of attention and working-memory functions.

Delay discounting, defined as the preference for a small immediate reward over a larger delayed one, serves as an index of impulsivity. The DDT procedures followed previous protocols as described in the prior referenced articles by de la Peña et al.). Testing was conducted in standard operant chambers (Coulbourn Instruments, Allentown, PA) equipped with two levers, chamber and magazine lights, and a pellet dispenser. Prior to tests, rats were food restricted (to increase their motivation to work for food delivery) and trained to press a lever for a contingent sucrose pellet reward (45 mg, TestDiet, IN, USA). During 1-week training (30 min/day), pressing the “small and immediate” lever (L1) delivered one pellet, while the “large and delayed” lever (L5) delivered five pellets. The chamber light illuminated for 1 s before pellet delivery, followed by a 25-s timeout signaled by the magazine light. Once rats selected L5 on at least two-thirds of trials, the testing phase began. Testing followed the same procedure except that delays (0, 10, and 30 s; 3 consecutive days) were imposed before L5 reward delivery. During the delay periods, the chamber light remained illuminated, and any additional lever presses were recorded but not reinforced. The 30-min DDT session commenced 20 min after intraperitoneal administration of vehicle or drug. Total responses on both L5 and L1 levers were recorded for analysis. To maintain motivation for food reinforcement, rats remained on restricted diets throughout the study. From the DDT data, the percentage choice for the large (L5) reinforcer was calculated as an index of impulsive choice (Winstanley et al., 2006), while responses on the L1 lever during time-out periods, when no reward was available, served as a measure of impulsive action (Zeeb et al., 2016).

In previous DDT studies described in the de la Peña references previously referenced and incorporated herein, consistent impulsive-like behavior has been demonstrated in SHR/NCrl and Wistar rats, relative to the WKY/NCrl, genetic control strain for the SHR/NCrl. As these baseline strain differences have been well established, WKY/NCrl rats were not included in the present study, allowing us to focus on the main objective of examining the effects of novel D4R-targetting drugs on impulsivity in strains with modifiable behavioral phenotypes, while also adhering to the principle of reduction in animal research by minimizing unnecessary replication of known findings.

Data were analyzed using two-way ANOVA followed by Dunnett's or Šidák post hoc tests where appropriate. Dunnett's test was used to compare each treatment group against the control, while Šidák's test was applied for pairwise comparisons among multiple groups. Statistical analyses were conducted using GraphPad Prism Version 9.5 software (San Diego, CA, USA). Results from the above analyses were presented as the means±S.E.M. Ap value of <0.05 was regarded as significant.

### Compound 15 (FMJ-01-38), but not Compound 17 (FMJ-01-54) Reduced Locomotor Hyperactivity in SHR/NCrl Rats

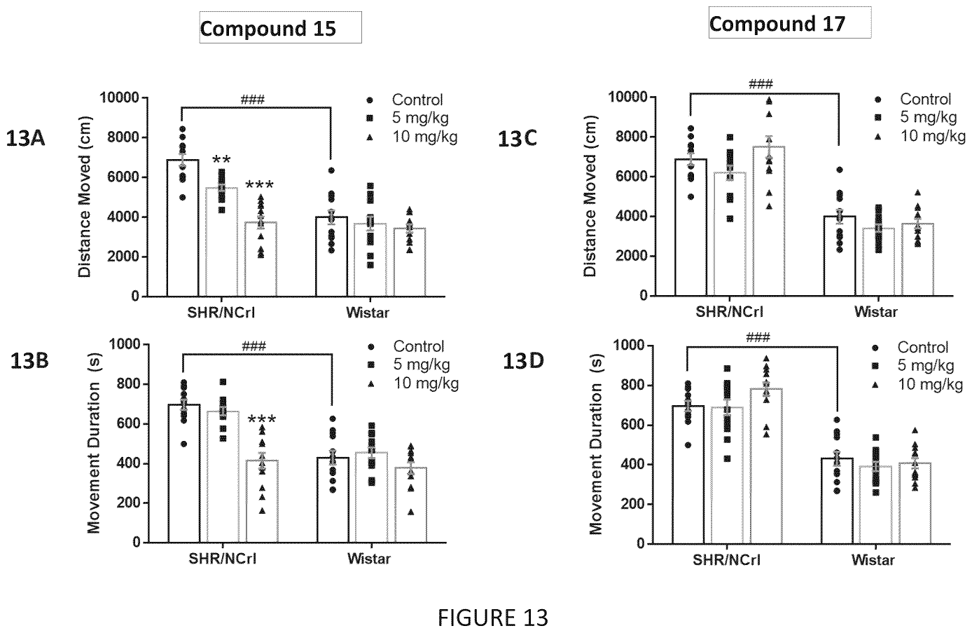

FIGS. 13A-13B show the effects of high efficacy D4R partial agonist Compound 15 (FMJ-01-38) on the locomotor activity of SHR/NCrl and Wistar rats. A two-way ANOVA of the 30-min total distance moved revealed significant main effects of strain [F (1,11)=148.20, p<0.001], drug treatment [F (2,22)=25.50, p<0.001], and a strain×treatment interaction [F (2,22)=8.37, p<0.01] (FIG. 13A). Post hoc comparisons indicated that SHR/NCrl rats exhibited higher locomotor activity than Wistar rats (p<0.001). Moreover, Compound 15 treatment produced a dose-dependent (5-10 mg/kg, i.p.) reduction in locomotor activity in SHR/NCrl rats, with no significant effects in Wistar rats (FIG. 13A). A two-way ANOVA of movement duration similarly revealed significant main effects of strain [F (1,11)=150.0, p<0.001], drug treatment [F (2,22)=22.0, p<0.001], and a strain×treatment [F (2,22)=8.43, p<0.01] interaction. Post hoc analysis showed that SHR/NCrl rats had longer movement durations than Wistar rats (p<0.001), and that Compound 15 significantly reduced movement duration in SHR/NCrl rats only at the 10 mg/kg (i.p.) dose (p<0.001, FIG. 13B).

FIGS. 13C-13D depict the effects of D₄R antagonist Compound 17 (FMJ-01-54) on locomotor activity in the same strains. Two-way ANOVA of total distance moved revealed a significant main effect of strain [F (1,11)=76.71, p<0.001], and drug treatment [F (2,22)=3.55, p<0.05) but no interaction between strain and drug treatment [F (2,22)=1.09, p=0.35, FIG. 13C]. Analysis of movement duration showed a significant main effect of strain [F (1,11)=73.86, p<0.001], but no effect of drug treatment [F (2,22)=2.41, p=0.11, FIG. 13D]. Consistent with these findings, SHR/NCrl rats had higher locomotor activity than Wistar rats (p<0.001), whereas Compound 17 (FMJ-01-54) produced no detectable effects on either total distance moved or movement duration in either strain across doses (FIG. 13D).

### Compound 15 (FMJ-01-38) and Compound 17 (FMJ-01-54) Improved Spontaneous Alternation Behavior in SHR/NCrl Rats

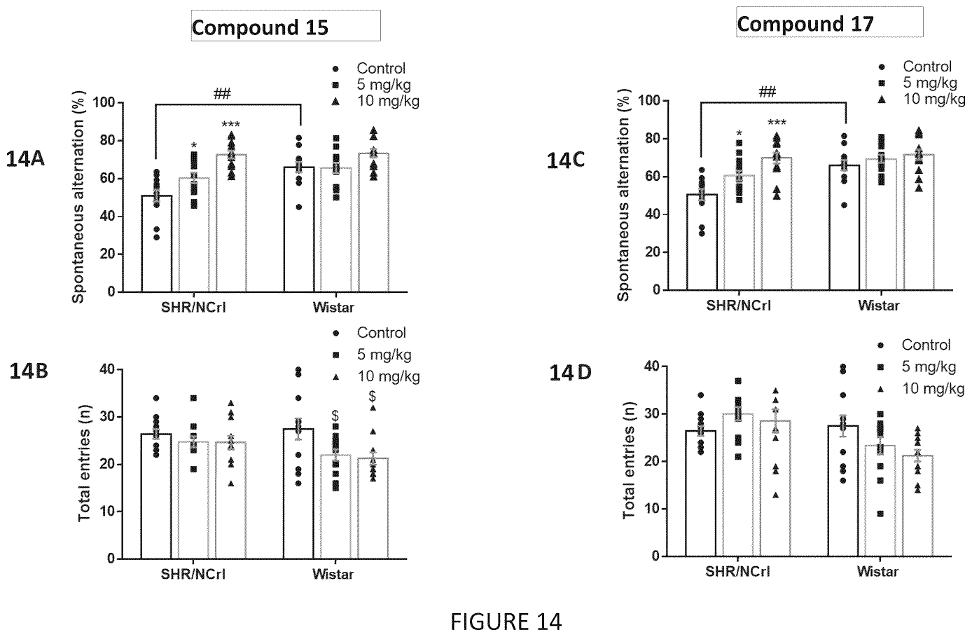

In line with our previous findings, SHR/NCrl rats showed impaired spontaneous alternation behavior compared with Wistar rats (FIGS. 14A, 14C). A two-way ANOVA of the percentage of spontaneous alternation revealed significant main effects of strain [F (1,11)=10.08, p<0.01] and drug treatment [F (2,22)=17.44, p<0.001], but no significant strain×treatment interaction [F (2,22)=3.02, p=0.06]. Post hoc analysis showed that Compound 15 (5-10 mg/kg, i.p.) dose-dependently improved spontaneous alternation behavior in SHR/NCrl rats (FIG. 14A). Analysis of total arm entries revealed significant main effects of strain [F (1,11)=8.50, p<0.05] and drug treatment [F (2,22)=4.54, p<0.05], with no significant strain×treatment interaction [F (2,22)=1.39, p=0.27] (FIG. 14B). Post hoc comparisons indicated that Compound 15 (FMJ-01-38) significantly reduced total arm entries in Wistar rats at both 5 mg/kg and 10 mg/kg doses (p<0.05).

Regarding Compound 17 (FMJ-01-54), two-way ANOVA of spontaneous alternation data revealed significant main effects of strain [F (1,11)=20.93, p<0.001] and drug treatment [F (2,22)=13.52, p=0.001], with no significant strain×treatment interaction [F (2,22)=2.09, p=0.14]. Post hoc analysis showed that Compound 17 (FMJ-01-54) (5-10 mg/kg, i.p.) dose-dependently improved spontaneous alternation behavior in SHR/NCrl rats but not in Wistar rats (FIG. 14C). Two-way ANOVA of total arm entries for Compound 17 revealed a significant main effect of strain [F (1,11)=20.24, p<0.001], but no significant effects of drug treatment [F (2,22)=0.54, p=0.58] or strain×treatment interaction [F (2,22)=3.30, p=0.55]. Consistent with these findings, Compound 17 did not significantly alter total arm entries in either SHR/NCrl or Wistar rats at any administered dose (FIG. 14D).

### Compound 15 (FMJ-01-38) and Compound 17 (FMJ-01-54) Enhanced Novel Object Preference in SHR/NCrl Rats

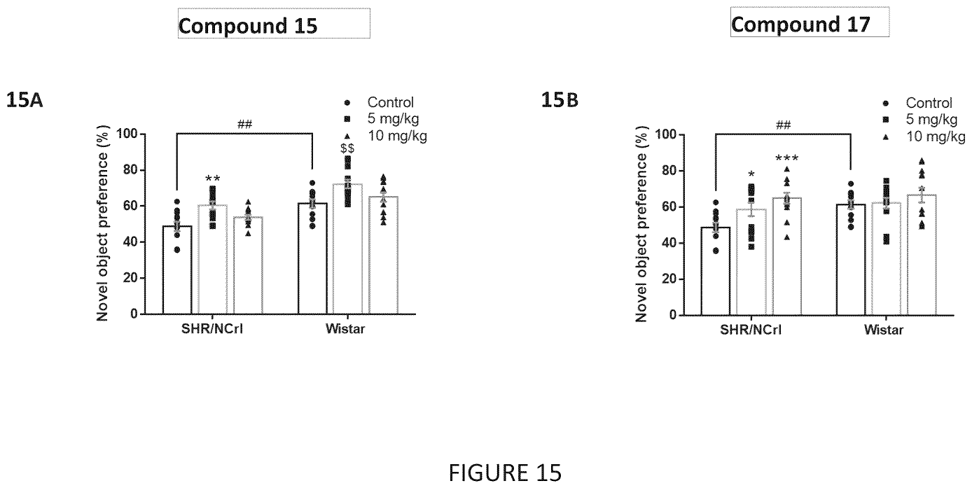

FIG. 15 shows the percentage of novel object preference in SHR/NCrl and Wistar rats and the effects of Compounds 15 and 17. For Compound 15 (FIG. 15A), two-way ANOVA of the % novel object preference revealed significant main effects of strain [F (1, 11)=71.74, p<0.001] and drug treatment [F (2, 22)=13.50, p<0.001], with no significant strain×treatment interaction [F (2,22)=0.05, p=0.95]. As expected, SHR/NCrl rats showed lower preference for the novel object than Wistar rats (p<0.001), consistent with impaired recognition memory and inattention. Treatment with Compound 15 (5 mg/kg, i.p.) significantly increased (p<0.01) novel object preference in SHR/NCrl rats. Interestingly, the same dose also enhanced novel object preference in Wistar rats (p<0.01), suggesting improvement of object recognition/attention in the normal rat strain.

For Compound 17 (FIG. 15B) two-way ANOVA likewise showed significant main effects of strain [F (1,11)=14.15, p<0.01] and drug treatment [F (2,22)=5.38, p<0.05) but no significant interaction between strain and treatment [F (2,22)=3.24, p=0.06]. Compound 17 increased the % novel object preference in SHR/NCrl rats in a dose-dependent manner (5-10 mg/kg, i.p.) and did not produce significant effects in Wistar rats.

### Compound 15 (FMJ-01-38), but not Compound 17 (FMJ-01-54) Reduced Impulsivity in SHR/NCrl Rats

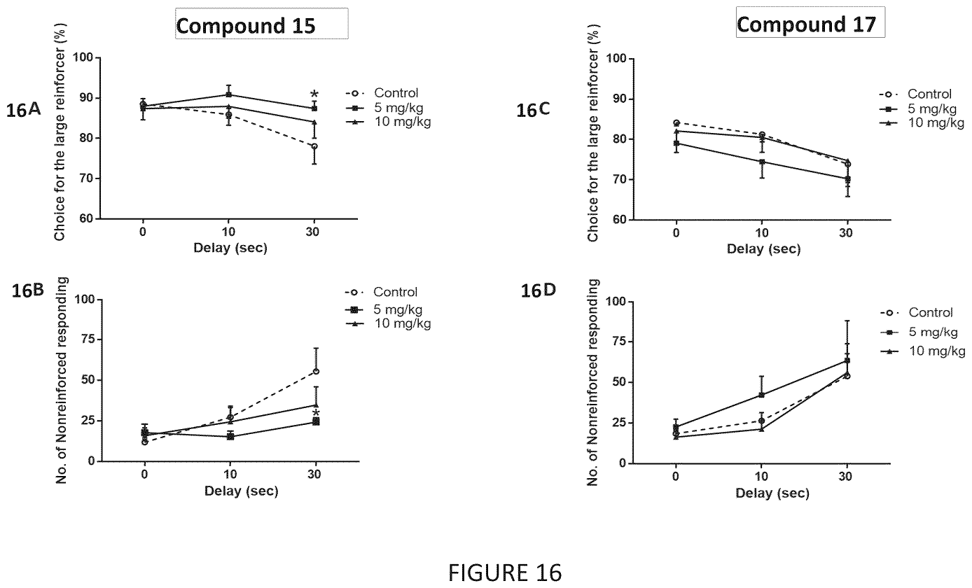

Vehicle-treated SHR/NCrl rats showed a reduced percentage of choice for the large, delayed reinforcer, indicating greater impulsive choice (FIG. 16A, 16C), and an increased frequency of lever presses for the small, immediate reward during the delay period, when such responses were nonreinforced, indicating impulsive action (FIG. 16B, 16D). These effects were most evident when the delay to reward delivery was increased to 30 seconds (FIGS. 16A-16D). At this 30-second delay, Compound 15 (5 mg/kg, i.p.) significantly improved both impulsive choice and impulsive action in SHR/NCrl rats (p<0.05 for both; FIGS. 16A-16B). In contrast, Compound 17 produced no significant effects on either parameter in SHR/NCrl rats.

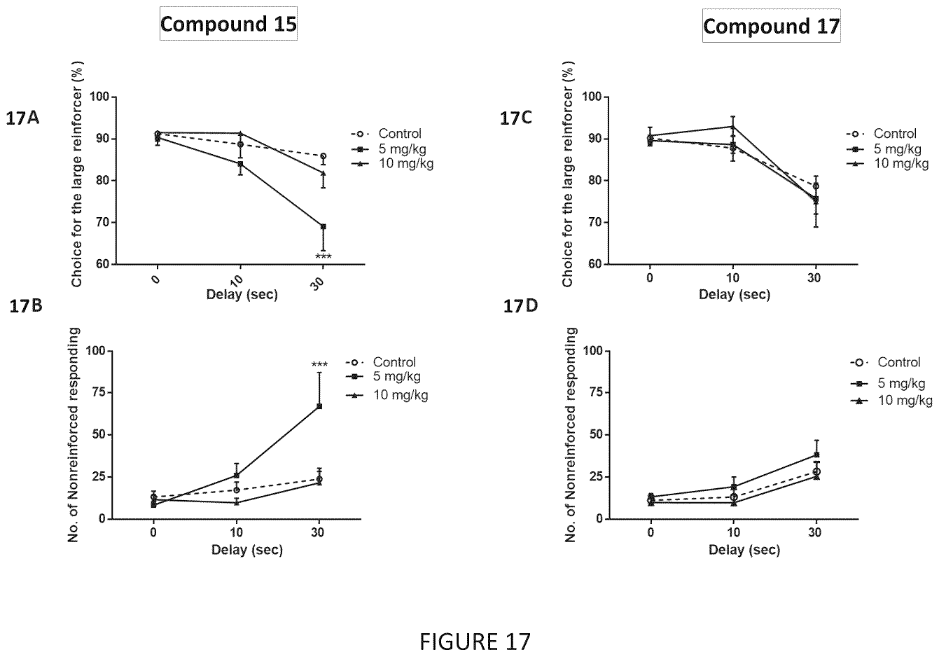

As shown in FIG. 17, control-treated Wistar rats also exhibited impulsive-like behavior, characterized by a decrease in % choice for the larger, delayed reinforcer (FIG. 17A, 17C) and increased lever pressing during the delay interval (FIG. 16B, 16D) at the 30-second delay. However, in contrast to the SHR/NCrl results, Compound 15 (5 mg/kg, i.p.) further increased impulsive choice and impulsive action in Wistar rats (p<0.05; FIGS. 17A-17B). Compound 17 treatment did not significantly alter impulsive choice or action in Wistar rats.

This study examined the effects of two novel dopamine D4 receptor (D4R) ligands, the high-efficacy partial agonist Compound 15 and the antagonist Compound 17, in SHR/NCrl rats, a validated model of ADHD, and Wistar rats across four behavioral assays assessing locomotor activity (open field test), attention and working memory (Y-maze test), recognition memory (novel object preference), and impulsivity (delay discounting task). In adolescent SHR/NCrl rats, Compound 15 dose-dependently (5-10 mg/kg, i.p.) reduced locomotor hyperactivity and improved spontaneous alternation behavior; at 5 mg/kg, it further enhanced novel-object preference and reduced impulsive choice and action in the delay-discounting task, indicating amelioration of ADHD-like behaviors and improved cognitive performance. In contrast, the D4R antagonist Compound 17 produced dose-dependent improvements in Y-maze and novel-object preference performance but did not reduce locomotor hyperactivity or impulsivity in SHR/NCrl rats, suggesting a selective cognitive-enhancing effect. In Wistar rats, Compound 15 enhanced novel-object preference only at the 5 mg/kg dose, whereas Compound 17 produced no significant behavioral effects.

Previous studies have demonstrated the cognitive-enhancing effects of partial D4R activation as well as potential mechanisms underlying attention- and impulse control-improving actions of D4R partial agonist. The present findings extend these observations by showing that Compound 15 not only enhances cognition in unimpaired animals but, importantly, attenuates hyperactivity, inattention, and impulsivity in the SHR/NCrl model of ADHD. As previously noted, SHR rats exhibit markedly reduced D4R gene expression and protein synthesis in the PFC accompanied by a hypoactive dopaminergic system characterized by reduced vesicular dopamine storage and release. Consistent with previous reports that full D4R agonists (e.g., ABT-724, A-412997) alleviated SHR hyperactivity and cognitive deficits, the behavioral improvements observed with Compound 15 likely reflect restoration of PFC inhibitory regulation via submaximal D4R activation and compensation for reduced dopaminergic tone, thereby stabilizing cortico-striatal signaling known to be dysregulated in ADHD. In SHR rats, where PFC D4R density is reduced, Compound 15 likely acts predominantly as an agonist, engaging Gi/o-coupled signaling and modulating downstream cAMP-PKA and ERK-MAPK pathways implicated in attention and executive control. Through these mechanisms, Compound 15 may restore excitatory-inhibitory balance in the PFC, normalize cortical output, and reduce behavioral manifestations of hyperactivity, inattention, and impulsivity in SHR/NCrl rats. The precise cellular and molecular mechanisms mediating these effects warrant further experimental investigation. In contrast, in Wistar rats with presumably intact dopaminergic tone, Compound 15 would typically be expected to act as a functional antagonist, producing limited or no behavioral effects consistent with the state-dependent nature of partial agonists. Interestingly, Compound 15 further increased impulsivity in Wistar rats, suggesting that its antagonistic effect may have disrupted PFC dopaminergic balance, thereby impairing inhibitory control and promoting impulsive behavior. Nonetheless, the improvement of novel object preference in Wistar rats suggests that D4R activation may also facilitate cognition beyond ADHD-related pathology, reflecting a broader modulatory role of D4R signaling in regulating cognitive functions.

Aside from partial agonists, the therapeutic potential of D4R blockers has also been reported. Selective D4R antagonists such as L-745,870 and U-101,958 reduced hyperactivity in dopamine-depleted or lesion-based ADHD models without affecting locomotor activity in controls. In this study, however, Compound 17 did not attenuate SHR/NCrl rats' hyperactivity, likely reflecting model-specific or drug-related differences. Notably, 6-OHDA lesions broadly affect dopaminergic projections and are not restricted to the D4R subtype. Moreover, previously used D4R antagonists exhibit off-target serotonergic and sigma receptor activity. Nonetheless, Compound 17 improved attention and recognition memory in SHR/NCrl rats, representing, to our knowledge, the first evidence of a D4R antagonist enhancing cognition in a validated ADHD model. Notably, L-745,870 has been shown to improve working memory in other studies, with an inverted U-shaped dose-response relationship, enhancing performance only in subjects with lower baseline cognitive function. The selective effects of Compound 17 in SHR/NCrl, but not in Wistar rats, are consistent with this pattern/observation. Furthermore, Compound 17 did not affect locomotion or impulsivity, suggesting that D4R antagonism exerts selective cognitive-enhancing effects that emerge primarily under conditions of dopaminergic dysfunction. Further studies are needed to elucidate the molecular and neurophysiological mechanisms underlying the effects of Compound 17 on ADHD-related cognitive and attentional/working memory deficits.

From a translational perspective, these findings suggest the potential of D4R ligands as novel, non-stimulant therapeutics for ADHD. Notably, Compound 15 improved ADHD-like behavioral deficits in SHR/NCrl rats while producing minimal effects in control animals, consistent with the concept of state-dependent or pathology-selective therapy that normalizes dysregulated neural function without altering normal brain activity. Unlike psychostimulants that globally elevate catecholamine levels (Dela Peña et al., 2015c), D4R partial agonists such as Compound 15 may provide targeted modulation of fronto-striatal circuits, thereby reducing abuse liability and peripheral adverse effects. Moreover, by producing submaximal receptor activation, D4R partial agonists such as Compound 15 may confer therapeutic benefits while reducing the behavioral and physiological effects typically observed with full D4R stimulation. In parallel, the selective cognitive benefits of Compound 17 suggest that D4R antagonism could complement partial agonism by improving executive or attentional function through distinct mechanisms. Collectively, these results strengthen the therapeutic relevance of D4R modulation and warrant further investigation into receptor-state dynamics, signaling mechanisms, and long-term adaptations underlying D4R-targeted interventions.

The present study has a few limitations that warrant consideration. First, the behavioral effects of D4R drugs were assessed at only two doses, necessitating broader dose-response studies to better define their therapeutic and adverse profiles. Second, as ADHD is a chronic disorder requiring long-term treatment, future work should evaluate the sustained efficacy and safety of these compounds under chronic dosing to detect potential neuroadaptive changes. Third, the study was limited to male adolescent rats, precluding assessment of sex- or age-dependent differences in D4R function. Fourth, although SHR/NCrl rats are a validated ADHD model, they cannot fully capture the complexity of human ADHD symptoms. Finally, the study relied primarily on behavioral parameters without direct neurochemical or molecular correlates; thus, the mechanistic basis of the observed improvements, such as modulation of PFC D4R signaling, dopamine release, or downstream signaling cascades, remains to be elucidated.

In conclusion, this study demonstrated that the D4R-targeting compounds Compound 15 and Compound 17 produced distinct yet significant behavioral effects in the SHR/NCrl model of ADHD. These findings support the therapeutic potential of D4R modulation, suggesting that both partial agonism and antagonism may confer symptom-selective benefits in ADHD through distinct mechanisms. Overall, the results suggest D4 receptor as a promising molecular target for developing safer and more specific non-stimulant treatments for ADHD.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the pending claims. Although specific terms are employed herein, they are used in a generic and descriptive sense only and not for purposes of limitation.

What is claimed is:

1. A compound of Formula 1: 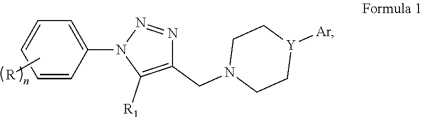
wherein:
R is a halogen, hydroxyl, C1-6 alkyl or C1-6 alkoxy, wherein the C1-6 alkyl or C1-6 alkoxy can be optionally substituted with one or more substituents selected from the group consisting of halogen, optionally substituted C1-6 alkyl, optionally substituted C1-6 alkenyl, OR₂, and N(R₂)₂;
n is an integer from 0 to 5;
R₁ is H, C1-6 alkyl, aryl, or heteroaryl, wherein the alkyl, aryl, or heteroaryl is optionally substituted with one or more substituents selected from the group consisting of halogen, optionally substituted C1-6 alkyl, optionally substituted C1-6 alkenyl, OR₃, and N(R₂)₂;
Y is N or CH;
Ar is aryl or heteroaryl, wherein the aryl or heteroaryl is optionally substituted with one or more substituents selected from the group consisting of halogen, optionally substituted C1-6 alkyl, optionally substituted C1-6 alkenyl, OR₂, and N(R₂)₂;
R₂ is selected from H and C1-6 alkyl; and
R₃ is selected from H, optionally substituted C1-6 alkyl, and optionally substituted C1-6 aralkyl.

2. The compound of claim 1, wherein n=0.

3. The compound of claim 1, wherein n=1 and R is C1-6 alkyl.

4. The compound of claim 1, wherein Y is N.

5. The compound of claim 1, wherein Ar is an optionally substituted phenyl ring.

6. The compound of claim 1, wherein Ar is an optionally substituted pyridine ring.

7. The compound of claim 6, wherein the optionally substituted pyridine ring is an optionally substituted 2-pyridinyl ring.

8. The compound of claim 1, wherein Ar is an optionally substituted naphthyl group.

9. The compound of claim 1, wherein Ar comprises no substituent.

10. The compound of claim 1, wherein Ar comprises one substituent.

11. The compound of claim 1, wherein Ar comprises a Cl or CH₃ substituent.

12. The compound of claim 1, wherein R₁ is H.

13. The compound of claim 1, wherein R is CH₃, Y is N, R₁ is H, and Ar is a substituted pyridine ring.

14. The compound of claim 1, wherein R₁ is

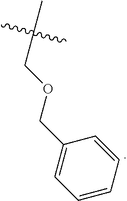 [SMILES: *COCc1ccccc1]

15. The compound of claim 1, selected from the group consisting of:

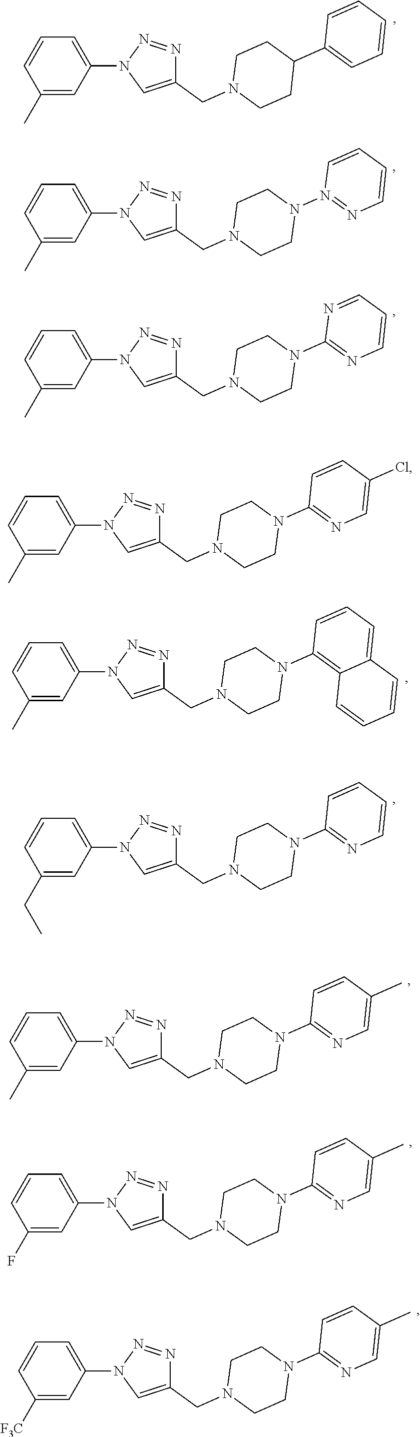

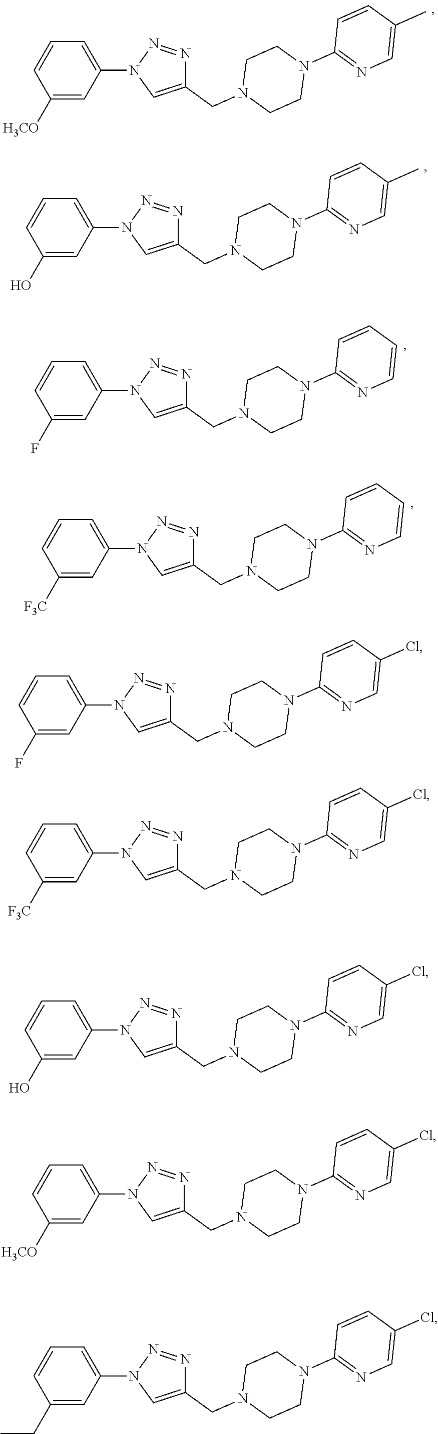

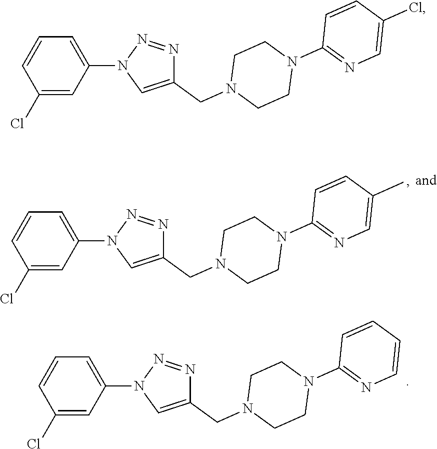 [SMILES: Clc1ccc(N2CCN(Cc3cn(-c4cccc(Cl)c4)nn3)CC2)nc1 ; Cc1ccc(N2CCN(Cc3cn(-c4cccc(Cl)c4)nn3)CC2)nc1 ; Clc1cccc(-n2cc(CN3CCN(c4ccccn4)CC3)nn2)c1]

16. The compound of claim 1, selected from the group consisting of:

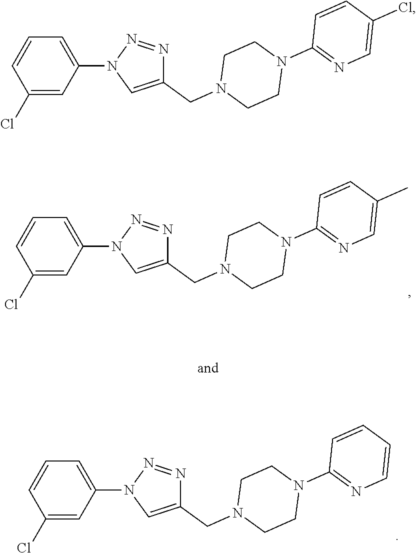 [SMILES: Clc1ccc(N2CCN(Cc3cn(-c4cccc(Cl)c4)nn3)CC2)nc1 ; Cc1ccc(N2CCN(Cc3cn(-c4cccc(Cl)c4)nn3)CC2)nc1 ; Clc1cccc(-n2cc(CN3CCN(c4ccccn4)CC3)nn2)c1]

17. A pharmaceutical composition comprising the compound of claim 1 and one or more pharmaceutically acceptable carriers.

18. A method for treating a disease or disorder associated with the central nervous system, comprising administering a therapeutically effective amount of the compound of claim 1.

19. The method of claim 18, wherein the disease or disorder is a cognitive deficit associated with neuropsychiatric disorders, e.g., schizophrenia and/or attention deficit/hyperactivity disorder (ADHD).

20. The method of claim 18, wherein the disease or disorder is L-DOPA-induced dyskinesias or an impulse-control disorder, e.g., substance abuse disorders (e.g., cocaine addiction or opioid addiction), eating disorders, and pathological gambling.
